(19) United States
(12) Patent Application Publication
(10) Pub. No.: US 2016/0103044 A1
(43) Pub. Date: Apr. 14, 2016

(54) DEVICES AND METHODS FOR DIAGNOSING, PROGNOSING, OR THERANOSING A CONDITION BY ENRICHING RARE CELLS

(71) Applicant: GPB Scientific, LLC, Richmond, VA (US)

(72) Inventor: Anne R. Kopf-Sill, Portola Valley, CA (US)

(21) Appl. No.: 14/717,626

(22) Filed: May 20, 2015

Related U.S. Application Data
(63) Continuation of application No. 13/906,206, filed on May 30, 2013, now abandoned.
Application No. 13/906,206 is a continuation of application No. 12/595,950, filed on May 27, 2010, now abandoned.
(60) Provisional application No. 60/912,143, filed on Apr. 16, 2007.
(60) Provisional application No. 60/912,147, filed on Apr. 16, 2007.
(60) Provisional application No. 60/912,149, filed on Apr. 16, 2007.

(51) Int. Cl. G01N 1/31; G01N 33/574

(52) U.S. Cl. CPC G01N 1/31; G01N 33/57484

(57) ABSTRACT

The invention encompasses methods and devices for diagnosing, theranosing, or prognosing a condition in a patient by enriching a sample in rare cells. The devices can be a microfluidic device comprising an array of obstacles and one or more binding moieties. The devices and methods can allow for enrichment of cells based on size and affinity, recovery of cells in locations on the microfluidic device, release of cells from the microfluidic device, flow of sample through the microfluidic device, and retention of rare cells from a sample obtained from a patient having a condition.

### CROSS-REFERENCE TO RELATED APPLICATIONS

This application is a Continuation Application which claims the benefit of U.S. application Ser. No. 12/595,950, filed May 27, 2010; which is a national stage application of PCT/US08/60546, filed Apr. 16, 2008, which claims the benefit of U.S. Provisional Application No. 60/912,147, filed Apr. 16, 2007, U.S. Provisional Application No. 60/912,143, filed Apr. 16, 2007, and U.S. Provisional Application No. 60/912,149, filed Apr. 16, 2007, all of which are hereby incorporated by reference.

### TECHNICAL FIELD

The invention is related to medical diagnostics and methods for diagnosing, prognosing, or theranosing a condition in a patient.

### BACKGROUND

Cancer is a disease marked by the uncontrolled proliferation of abnormal cells. In normal tissue, cells divide and organize within the tissue in response to signals from surrounding cells. Cancer cells do not respond in the same way to these signals, causing them to proliferate and, in many organs, form a tumor. As the growth of a tumor continues, genetic alterations may accumulate, manifesting as a more aggressive growth phenotype of the cancer cells. If left untreated, metastasis, the spread of cancer cells to distant areas of the body by way of the lymph system or bloodstream, may ensue. Metastasis results in the formation of secondary tumors at multiple sites, damaging healthy tissue. Most cancer death is caused by such secondary tumors.

Despite decades of advances in cancer diagnosis, prognosis and therapy, many cancers are not diagnosed, prognosed or treated properly. As one example, most early-stage lung cancers are asymptomatic and are not detected in time for curative treatment, resulting in an overall five-year survival rate for patients with lung cancer of less than 15%. However, in those instances in which lung cancer is detected and treated at an early stage, the prognosis is much more favorable. As another example, breast cancer is detected in a patient and then subjected to a therapeutic treatment using monoclonal antibodies. However, the patient doesn't respond to the therapeutic treatment.

Therefore, there exists a need to develop new methods and devices for diagnosis, prognosis, and theranosis of cancer.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

In one aspect of the invention, a microfluidic device comprises an array of obstacles including a first subarray of obstacles and a second subarray of obstacles that are fluidly connected and positioned such that a fluid medium introduced to an inlet of the microfluidic device passes sequentially through the first subarray then the second subarray before exiting through an outlet of the microfluidic device; wherein the first subarray or the second subarray of obstacles is functionalized with one or more sets of one or more binding moieties.

The sets of one or more binding moieties can include two or more binding moieties. The first subarray and the second subarray of obstacles can be functionalized with one or more sets of one or more binding moieties. The obstacles can be fixed to the microfluidic device.

The microfluidic device comprising a first subarray of obstacles and a second subarray of obstacles can further comprise a first set of one or more binding moieties functionalized in a first region of the first subarray and a second set of one or more binding moieties functionalized in a second region of the first subarray. The microfluidic device comprising a first subarray of obstacles and a second subarray of obstacles can further comprise a first set of one or more binding moieties functionalized in a first region of the second subarray and a second set of one or more binding moieties functionalized in a second region of the second subarray. The first set of one or more binding moieties and the second set of one or more binding moieties include two or more binding moieties. The first region can be distinct from the second region.

The first subarray can have a first average gap length between adjacent obstacles and the second subarray can have a second average gap length between adjacent obstacles, wherein the first average gap length is greater than the second average gap length. The second average gap length can be less than 8, 10, 12, 15, 17, 20, 24, 29, 35, or 42 microns.

A sample obtained from a patient can be contacted with the microfluidic device and one or more rare cells can be retained by the microfluidic device. 1, 5, or 20% of the one or more rare cells retained by the microfluidic device are retained in the first 30 rows of the second subarray of obstacles.

In another aspect of the invention, a method for diagnosing cancer comprises enumerating one or more enriched circulating tumor cells and fragments thereof using a bright field microscope. The enumerating can comprise staining the one or more enriched circulating tumor cells. The staining can include an indicator for a cancer marker. The cancer marker can be cytokeratin, EGFR, EpCAM, cadherin, mucin, or LAR. The cancer marker can be cytokeratin. The staining can include using a pan-cytokeratin antibody, a biotinylated secondary antibody, an avidin-biotinylated horseradish peroxidase complex, and diaminobenzidine tetrahydrochloride. The pan-cytokeratin antibody can be a mixture of monoclonal antibodies. The stain can include AE1/AE3 antibodies.

The enumerating can comprise measuring a total amount of stained area or measuring total intensity of stained area. The enumerating can comprise using a processor to enumerate the one or more enriched circulating tumor cells. The processor can enumerate the one or more enriched circulating tumor cells using an image of the enriched circulating tumor cells taken by a bright field microscope. The circulating tumor cells can be enriched based on affinity, cell size, cell shape, or cell deformability by flowing the cellular sample through a two-dimensional array of obstacles. The obstacles can be functionalized with at least one binding moiety. The staining includes using an indicator for determining a tissue of origin for the one or more enriched circulating tumor cells. The staining can include using an indicator for determining efficacy of a cancer therapeutic. The staining can include using a fluorescent dye. Enumerating the enriched one or more circulating tumor cells can comprise using a fluorescence microscope.

In one aspect of the invention, a method for diagnosing cancer comprises enumerating one or more enriched stem cells using a bright field microscope.

The invention provides for a kit comprising a microfluidic device for enriching rare cells and at least one immunochemical stain that is visualized using a bright field microscope that selectively binds enriched rare cells or fragments thereof. The immunochemical stain can include AE1/AE3. The immunochemical stain can specifically bind cytokeratin.

In another aspect of the invention, a method for enriching rare cells comprises a) flowing a sample including one or more rare cells through a first array of obstacles that selectively retains said rare cells; b) allowing said sample to remain in contact with said array of obstacles; and c) removing a portion of said sample.

The array of obstacles can be functionalized with one or more binding moieties, the array of obstacles form a network of gaps between obstacles, and/or the rare cells are epithelial cells or circulating tumor cells. The one or more binding moieties can be anti-EpCAM. The sample can remain in contact with said first array of obstacles for more than 0.5, 2, 5, 10, 15, 30, 60, or 120 minutes. The flow rate of sample through the first array of obstacles can be 0.1 mL/hr or less during step b). Allowing said sample to remain in contact with said array of obstacles can comprise incubating said sample with said array of obstacles. The first array of obstacles can form a network of gaps between adjacent obstacles, and further wherein the gaps can be between 1 and 300 microns in length.

The method for enriching rare cells comprising allowing the sample to remain in contact with said array of obstacles can further comprise flowing the portion of the sample removed in step c) through a second array of obstacles.

The method for enriching rare cells comprising allowing the sample to remain in contact with said array of obstacles can further comprise d) flowing the portion of the sample removed in step c) through said first array of obstacles.

The method for enriching rare cells comprising allowing the sample to remain in contact with said array of obstacles can further comprise e) repeating steps a), b), c) and d) at least one, two, or three times.

In one aspect of the invention, a method for determining if a subject has a critical concentration of circulating tumor cells comprises generating a sample test solution by adding a known number of discrete particles to a sample obtained from the subject, wherein each discrete particle comprises a circulating tumor cell antigen; contacting the sample test solution with a plurality of capture elements comprising a binding moiety that binds specifically to the circulating tumor cell antigen; and determining a number of discrete particles captured by the plurality of capture elements; determining a number of circulating tumor cells captured by the plurality of capture elements; determining if the subject has the critical concentration of circulating tumor cells; and reporting to the subject results of determining if the subject has the critical concentration of circulating tumor cells.

The subject has the critical concentration of circulating tumor cells can be based on a capture efficiency determined by the number of discrete particles captured by the plurality of capture elements and the known number of discrete particles added to the sample, the expected number of circulating tumor cells captured by the plurality of capture elements for a subject having the critical concentration, the number of circulating tumor cells captured by the plurality of capture elements, and the total volume of the sample contacted with the plurality of capture elements.

The capture elements can comprise an array of obstacles functionalized with said one or more binding moieties. The array of obstacles can be fixed to a microfluidic device and/or the array of obstacles form a network of gaps between adjacent obstacles that are between 5 and 300 microns in length.

The absence of circulating tumor cells captured by the plurality of capture elements can indicate that the likelihood that the subject has the critical concentration of circulating tumor cells is less than a diagnostic risk level. The diagnostic risk level can be less than 0.001, 0.01, or 0.1. The sample can be blood and the critical concentration can be between about 1 to 10, about 1 to 20, about 20 to 40, or about 40 to 100 cells per 10 mL of blood. The discrete particles can be agarose beads or dendrimers. The discrete particles can have an average size that is 0.5, 1, 2, 4, 5, or 10 microns larger or smaller than an average size of the circulating tumor cells captured by the plurality of capture elements. The discrete particles can be labeled with a first dye and the circulating tumor cells are labeled with a second dye. The first dye and second dye can have light absorption wavelengths or fluorescent light emission wavelengths that are separated by at least 5, 10, 20, 40, 50, 75, or 100 nm. The circulating tumor cell antigen can comprise EpCAM.

In another aspect of the invention, a microfluidic device adapted to enrich rare cells from a sample comprises one or more of the following features: a) an array of obstacles functionalized with binding moieties, wherein said array of obstacles comprises between 20 and 20,000 rows and between 10 and 1,000 columns of obstacles; b) an array of obstacles functionalized with binding moieties, wherein said array ob obstacles comprises at least 1000 obstacles; c) an array of obstacles functionalized with binding moieties that is adapted to process at least 0.5, 1, 1.5, 5, 10, 25, 500, or 1000 mL/hour of sample; d) an array of obstacles functionalized with binding moieties, wherein the binding moieties comprise two different binding moieties; e) an array of obstacles functionalized with binding moieties, wherein at least 50% of the surface area of the microfluidic device contacting the sample is functionalized with binding moieties; f) an array of obstacles functionalized with binding moieties, wherein the amount of surface area of the microfluidic device contacting the sample is at least 30, 50, 75, 100, 250, or 500 mm2; g) an array of obstacles enclosed by a chamber, wherein the chamber can hold at least 2, 5, 10, 25, 50, or 100 μL of fluid; h) an array of obstacles enclosed in a chamber, wherein at least 5%, 10%, 25%, 35%, 50%, or 65% of the interior volume of said chamber is occupied by said obstacles; i) an array of obstacles, wherein said array of obstacles comprises a first array of obstacles fluidly coupled to a second array of obstacles, and further wherein said first array of obstacles has a restricted gap dispersed in a uniform pattern and said second array of obstacles has a uniform pattern of obstacles and no restricted gap; j) an array of obstacle functionalized with one or more binding moieties, wherein the array of obstacles are fixed to the microfluidic device; or k) an array of obstacles functionalized with one or more binding moieties, a lid, and a port. The binding moieties can be anti-EpCAM or anti-EGFR.

In one aspect of the invention, a microfluidic device comprises an array of obstacles; and one or more binding moieties, wherein the device is configured to enrich at least one rare cell from a fluid sample from at least 10, 20, 25, or 50% of at least stage 1 of cancer patients without mechanically damaging said rare cell.

The microfluidic device does not need to comprise magnetic beads. The microfluidic device can further comprises a lid. The lid can be optically transparent, wherein said lid can be adapted and configured for an optical detection means positioned adjacent to or above said array of obstacles to analyze cells retained within said array. The array of obstacles can form a network of gaps between adjacent obstacles, and further wherein the gaps between adjacent obstacles are between 1 and 300 microns in length. The one or more binding moieties can include anti-EpCAM.

In another aspect of the invention, a method for diagnosing, theranosing, or prognosing cancer in a patient comprises obtaining a sample from said patient; flowing said sample through a microfluidic device adapted for retaining one or more rare cells in at least 5, 10, 20, 25, or 50% of patients having at least stage 1 of said cancer; and making a diagnosis, theranosis, or prognosis based on retained cells. The one or more rare cells can be not mechanically damaged by flowing said sample through the microfluidic device. The one or more rare cells can be circulating tumor cells or epithelial cells. The microfluidic device can comprise one or more binding moieties and/or an array of obstacles. The array of obstacles can form a network of gaps between adjacent obstacles, and further wherein the gaps between adjacent obstacles are between 1 and 300 microns in length. The one or more binding moieties can include anti-EpCAM.

In one aspect of the invention, a method for determining viability of a circulating tumor cell in a sample obtained from a subject comprises contacting the sample with a cell membrane-impermeable nucleic acid binding agent capable of being photoactivated; exposing the sample to a dose of light to photoactivate the nucleic acid binding reagent; capturing a circulating tumor cell from the sample; and detecting the presence or absence of the nucleic acid binding reagent in the nucleus of the captured circulating tumor cell, wherein the presence of the nucleic acid binding reagent indicates that the captured circulating tumor cell is not viable.

The circulating tumor cell can be captured using a microfluidic device comprising an array of obstacles and/or one or more binding moieties. The array of obstacles can form a network of gaps between adjacent obstacles, and further wherein the gaps between adjacent obstacles are between 1 and 300 microns in length.

In another aspect of the invention, a microfluidic device for enriching one or more rare cells from a fluid sample comprises an array of obstacles forming a network of gaps between adjacent obstacles; and

one or more binding moieties, wherein the one or more binding moieties are attached to said microfluidic device via a cleavable linker and selectively bind rare cells.

The gaps can be between 1 and 300 microns in length. The array of obstacles can be fixed and/or the one or more binding moieties are anti-EpCAM. The rare cells can be epithelial cells or circulating tumor cells. The cleavable linker can comprise a Neutravidin, avidin, or streptavidin protein attached to the microfluidic device and a biotin-polynucleotide-anti-EpCAM moiety. The cleavable linker can be cleaved by a DNase.

In one aspect of the invention, a device for diagnosing, theranosing, or prognosing a condition in a patient comprises a microfluidic device comprising an array of obstacles and one or more binding moieties that selectively retains one or more rare cells, wherein the microfluidic device is configured for flowing between about 7-1,500, 0.1-1,500, 1-1000, or 1.5-500 mL/hr of blood sample from said patient through said microfluidic device.

The one or more binding moieties can be anti-EpCAM. The one or more rare cells can be circulating tumor cells or epithelial cells. The microfluidic device can contain no more than 50, 100, or 200 μL, of said sample. The microfluidic device can comprise no more than one microfluidic device.

In one aspect of the invention, a method for diagnosing, theranosing, or prognosing a condition in a patient comprises flowing between about 7-1,500, 0.1-1,500, 1-1000, or 1.5-500 mL/hr of blood sample from said patient through a microfluidic device comprising an array of obstacles and one or more binding moieties that selectively retains one or more rare cells; and enriching in one or more rare cells.

The one or more binding moieties are anti-EpCAM. The one or more rare cells can be circulating tumor cells or epithelial cells. The microfluidic device can contain no more than 50, 100, or 200 μL, of said sample. The microfluidic device can comprise no more than one microfluidic device.

In one aspect of the invention, a device for enriching one or more rare cells from a sample obtained from a patient comprises a microfluidic device including a capture array of obstacles covered with binding moieties to selectively retain said rare cells and a separation array of obstacles covered with binding moieties to selectively retain said rare cells, wherein at least 1, 5, 10, 25, 50 or 75% of said rare cells are retained within at least the first 30 rows of said capture array of obstacles, and further wherein said sample is at least 50, 75, or 100 times greater than an interior volume of the microfluidic device.

The rare cells can be circulating tumor cells. The capture array of obstacles can be fluidly coupled to the separation array of obstacles and can be positioned such that the sample contacts said separation array of obstacles prior to contacting said capture array of obstacles. The capture array of obstacles can comprise a network of gaps with an average capture gap length between adjacent obstacles and the separation array of obstacles comprises a network of gaps with an average separation gap length between obstacles. The average capture gap length can be no more than 20 microns and the average separation gap length can be no less than 20 microns. The average capture gap length can be less than the average separation gap length. The binding moieties can comprise anti-EpCAM, anti-EGFR, anti-LAR, or anti-cytokeratin.

In another aspect of the invention, a method for enriching one or more rare cells from a sample obtained from a patient comprises flowing said sample through a microfluidic device including a capture array of obstacles covered with binding moieties to selectively retain said rare cells and a separation array of obstacles covered with binding moieties to selectively retain said rare cells, wherein at least 1, 5, 10, 25, 50 or 75% of said rare cells are retained within at least the first 30 rows of said capture array of obstacles, and further wherein said sample is at least 50, 75, or 100 times greater than an interior volume of the microfluidic device. The rare cells can be circulating tumor cells.

The method for enriching one or more rare cells from a sample by flowing said sample through a microfluidic device including a capture array and a separation array can further comprise analyzing the retained rare cells. The analyzing can comprise enumerating, labeling, or imaging said rare cells.

The method for enriching one or more rare cells from a sample by flowing said sample through a microfluidic device including a capture array and a separation array can further comprise diagnosing, theranosing, or prognosing said patient.

### BRIEF DESCRIPTION OF THE FIGURES

FIG. 1A: Depicted is a microfluidic device having a lid and removable threaded screw ports attached to the inlet and to the outlet.

FIG. 1B: Depicted is a cross-sectional view of the microfluidic device of FIG. 1A having a lid and removable screw ports, cut along line B-B of FIG. 1A.

FIG. 2: Depicted is a system of three microfluidic devices wherein two devices are configured to flow a single sample in parallel, and wherein the third micrfluidic device is configured to flow the sample in series through the device after the sample has flowed through the first two devices, whereby the outlets of the first two devices flow to the inlet of the third device, and wherein a peristaltic pump is adapted and configured to flow the sample through the system.

FIG. 3: Depicted is a zoomed-in view of a blood sample flowing through an array of obstacles in a microfluidic device having generally columnar obstacles and having at least two controlled gap sizes between adjacent obstacles.

FIG. 4: Depicted is a zoomed-in view of a blood sample flowing through an array of obstacles in a microfluidic device having generally columnar obstacles.

FIG. 5: Depicted is a zoomed-in view of a blood sample flowing through an array of obstacles in a microfluidic device having generally columnar obstacles.

FIG. 6: Depicted is a zoomed-in view of a blood sample flowing through an array of obstacles in a microfluidic device having generally columnar obstacles.

FIG. 7: Depicted is a zoomed-in view of a blood sample flowing through an array of obstacles in a microfluidic device having generally half-circular obstacles.

FIG. 8: Depicted is a zoomed-in view of a blood sample flowing through an array of obstacles in a microfluidic device having generally columnar obstacles and having at least two controlled gap sizes between adjacent obstacles.

FIG. 9: Depicted is a zoomed-in view of a blood sample flowing through an array of obstacles in a microfluidic device having generally columnar obstacles and having at least two controlled gap sizes between adjacent obstacles.

FIG. 10: Depicted is a zoomed-in view of a blood sample flowing through an array of obstacles in a microfluidic device having generally columnar obstacles and having at least two controlled gap sizes between adjacent obstacles.

FIG. 11: Depicted is a listing of markers.

FIG. 12: Depicted is a capture plot showing rare cells captured using a microfluidic device from a blood sample.

FIG. 13: Depicted is a capture plot showing rare cells captured using a microfluidic device from a blood sample.

FIG. 14: Depicted is a capture plot showing rare cells captured using a microfluidic device from a blood sample.

FIG. 15: Depicted is a capture plot showing rare cells captured using a microfluidic device from a blood sample.

FIG. 16: Depicted is a capture plot showing rare cells captured using a microfluidic device from a blood sample.

FIG. 17: Depicted is a capture plot showing rare cells captured using a microfluidic device from a blood sample.

FIG. 18: Depicted is a plot showing recovery of rare cells as a function of incubation time.

FIG. 19: Depicted is a slide showing an experimental outline for evaluating H1650, HT29, and T24 cell lines.

FIG. 20: Depicted are graphs showing relative levels of EpCAM in H1650, HT29, and T24 cells.

FIG. 21: Depicted is a graph showing size distribution of H1650, HT29, and T24 cells.

FIG. 22: Depicted is a table showing cells captured and capture efficiency by T7-anti-EpCAM, T7-anti-IgG, MA1-anti-EpCAM, and MA1-anti-IgG microfluidic chips.

FIG. 23: Depicted is a plot showing recovery of cells as a function of cell lines.

FIG. 24: Depicted is a plot showing recovery of cells as a function of chip type.

FIG. 25: Depicted is a plot showing recovery of cells as a function of chip type.

FIG. 26: Depicted is a plot showing the number of cells captured using anti-EpCAM chips divided by the number of cells captured using anti-IgG chips

FIG. 27: Depicted is a plot showing the number of cells captured using anti-EpCAM chips subtracted by the number of cells captured using anti-IgG chips.

FIG. 28: Depicted is a diagram showing the obstacle diameter and gap spacing in subarrays of a MA1 chip and two capture plots showing spatial localization of cells captured by MA1-anti-EpCAM and MA1-anti-IgG chips.

FIG. 29: Depicted are two capture plots showing spatial localization of cells captured by T7-anti-EpCAM and T7-anti-IgG chips.

FIG. 30: Depicted is a fluorescence microscope image showing spatial localization of cells captured by a MA1-anti-EpCAM chip.

FIG. 31: Depicted is a fluorescence microscope image showing spatial localization of cells captured by a MA1-anti-IgG chip.

### DETAILED DESCRIPTION OF THE INVENTION

Enrichment Devices

The present invention relates to various enrichment devices for enriching rare particles or particle fragments from a heterogeneous population of particles. In many instances, the application refers to cells, but it should be understood that cells are but one example of a particle that can be enriched using the devices herein and cellular components are also contemplated.

A rare cell can be a cell that is present as less than 10% of all cells in a sample. A rare cells can include, but is not limited to, a circulating tumor cell, an epithelial cell, a stem cell, an undifferentiated stem cell, a cancer stem cell, a bone marrow cell, a progenitor cell, a foam cell, a mesenchymal cell, an endothelial cell, an endometrial cell, a trophoblast, a cancer cell, an immune system cell (host or graft), a connective tissue cell, a bacteria, a fungi, or a pathogen (e.g., bacterial or protozoa).

An epithelial cell that is exfoliated from a solid tumor can be found in very low concentrations in the circulation of a patient with advanced cancer of the breast, colon, liver, ovary, prostate, and lung. Presence, quantity, and/or concentration of these cells in blood can be correlated with overall prognosis and/or response to therapy. Such an epithelial cell can be referred to as a circulating tumor cell. A circulating tumor cell can be an early indicator of tumor expansion or metastasis before the appearance of a clinical symptom.

A circulating tumor cell can be generally larger than most blood cells and can display a cell surface marker. Therefore, one useful approach for analyzing a circulating tumor cell in blood is to enrich one or more cells based on size, affinity, shape, and/or deformability resulting in a cell population enriched in one or more circulating tumor cells. In some embodiments of the invention, optimal enrichment selectively targets target cells or marker without non-specifically retaining non-target materials. These cell populations can then be subjected to further processing or analysis.

A sample can have a volume, for example, up to about 1 mL, up to about 2 mL, up to about 3 mL, up to about 4 mL, up to about 5 mL, up to about 7 mL, up to about 10 mL, up to about 20 mL, up about 50 mL, up to about 75 mL, up to about 100 mL, up to about 200 mL, up to about 500 mL, up to about 1000 mL, or up to about 1.5 L or more.

In some embodiments, the preparation system is adapted and configured to reduce the quantity of non-rare cells in the fluid sample prior to processing said sample through said chamber.

In some embodiment of the invention, one or more enucleated cells are removed from the sample prior to enrichment of one or more cells using size or affinity. In other embodiments of the invention, the sample is not centrifuged prior to enrichment of one or more cells using size or affinity.

In one non-limiting example, wherein said sample size is greater than 20 mL, the sample can be applied to a microfluidic device that separates cells based solely on size prior to application of the sample to an affinity based device. A sample is greater than 20 ml can be concentrated to reduce overall volume. For example, devices of the invention can be employed in order to concentrate a cellular sample of interest, e.g., a sample containing CTCs. By reducing the volume of buffer introduced into the fluid inlet so that this volume is significantly smaller than the volume of the cellular sample and, optionally, by eliminating some of the non-target cells based on size, concentration of target cells in a smaller volume results. This concentration step can, in some instances, improve the results of any downstream analysis performed.

Blood is a complex mixture of cells. The present invention provides devices for enriching rare cells from a complex mixture such as blood, or a solublized biopsy. Rare particles such as cells are enriched based on their unique properties such as size, shaper and/or deformability.

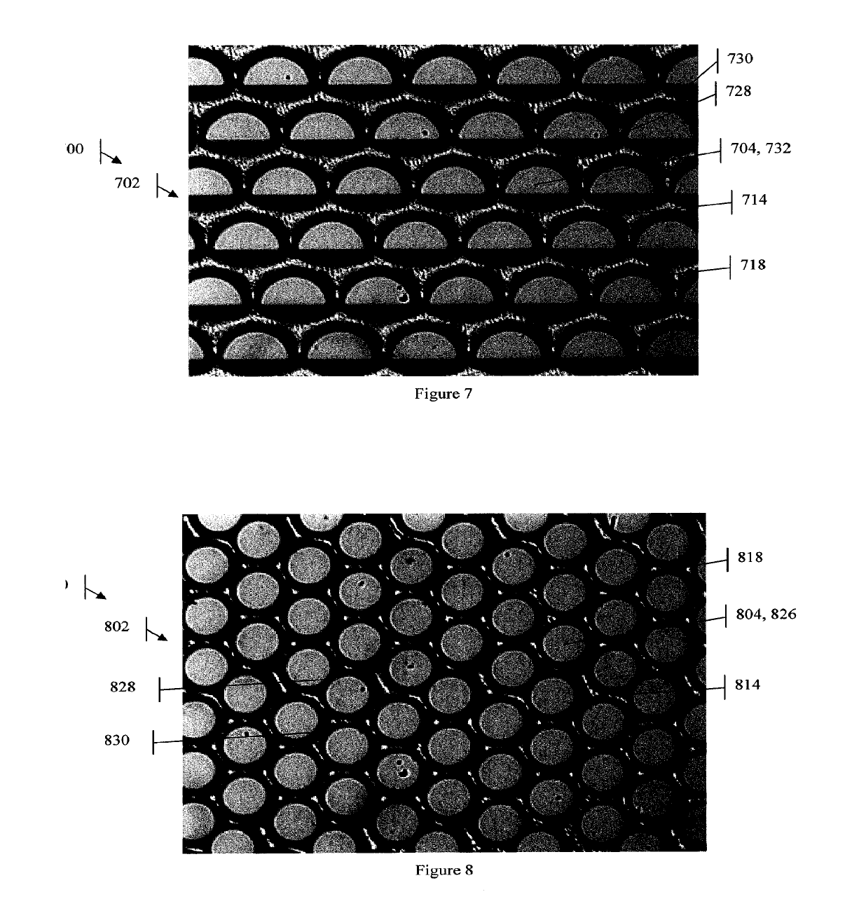

The devices herein are microfluidic and comprise an array of obstacles that extends in the flow direction and lateral direction (i.e., two dimensions). The obstacles form a network of microfluidic gaps and can be of any shape, including circle and half circle (FIG. 6-7).

The gaps can be configured to trap or capture cells larger than a critical size within the device, thus separating cells by size. For example, an enrichment device can be configured to retain cells having a hydrodynamic size greater than 12, 14, 16, 18, or even 20 microns.

Binding Moieties

The obstacles can be coupled to or covered with one or more binding moieties to selectively bind and retain subset of particles or cells of interest. Binding moieties include, but are not limited to, a nucleic acid (e.g., DNA, RNA, PNA, or oligonucleotide), a ligand, a protein (e.g. a receptor, a peptide, an enzyme, an enzyme inhibitor, an enzyme substrate, an antibody, an immunoglobulin (particularly an antibody or fragment thereof), an antigen, a lectin, a modified protein, a modified peptide, a biogenic amine, a complex carbohydrate, or a synthetic molecule.

Two different binding moieties can be on the same obstacles within an array or on different obstacles within the array or both. Also, two regions can have the same set of binding moieties, but in different concentration.

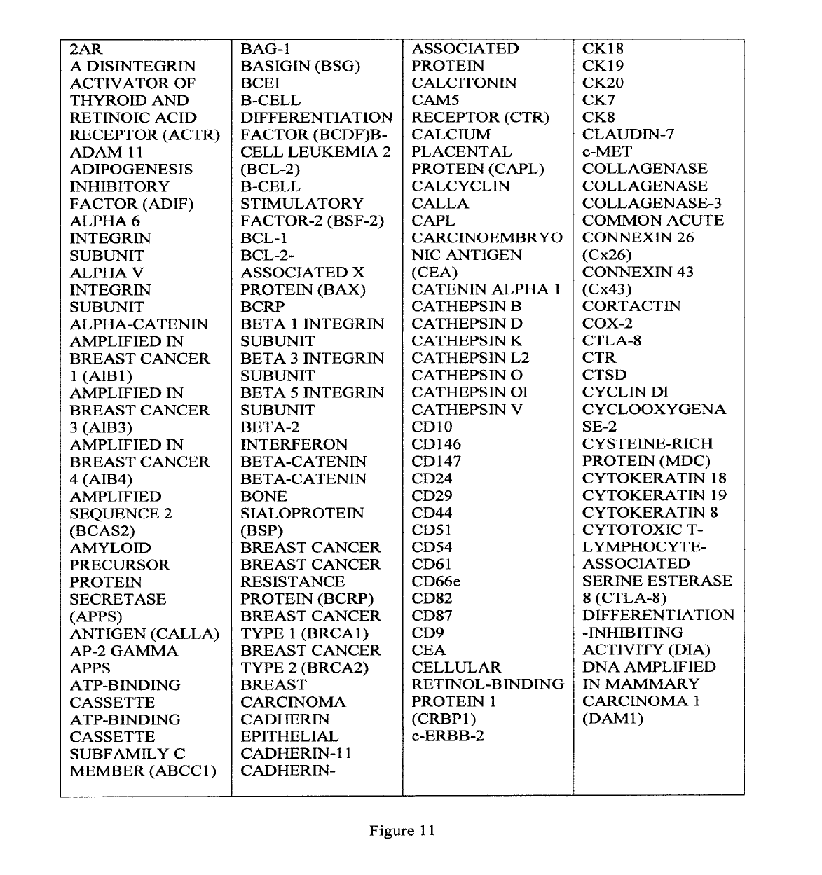

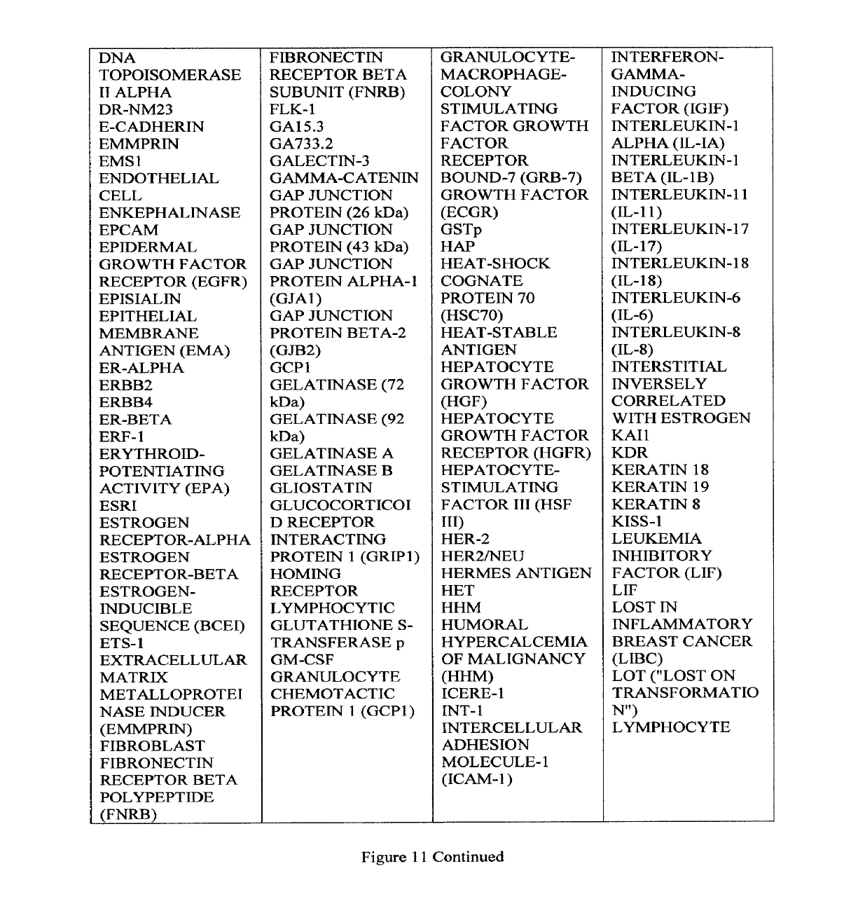

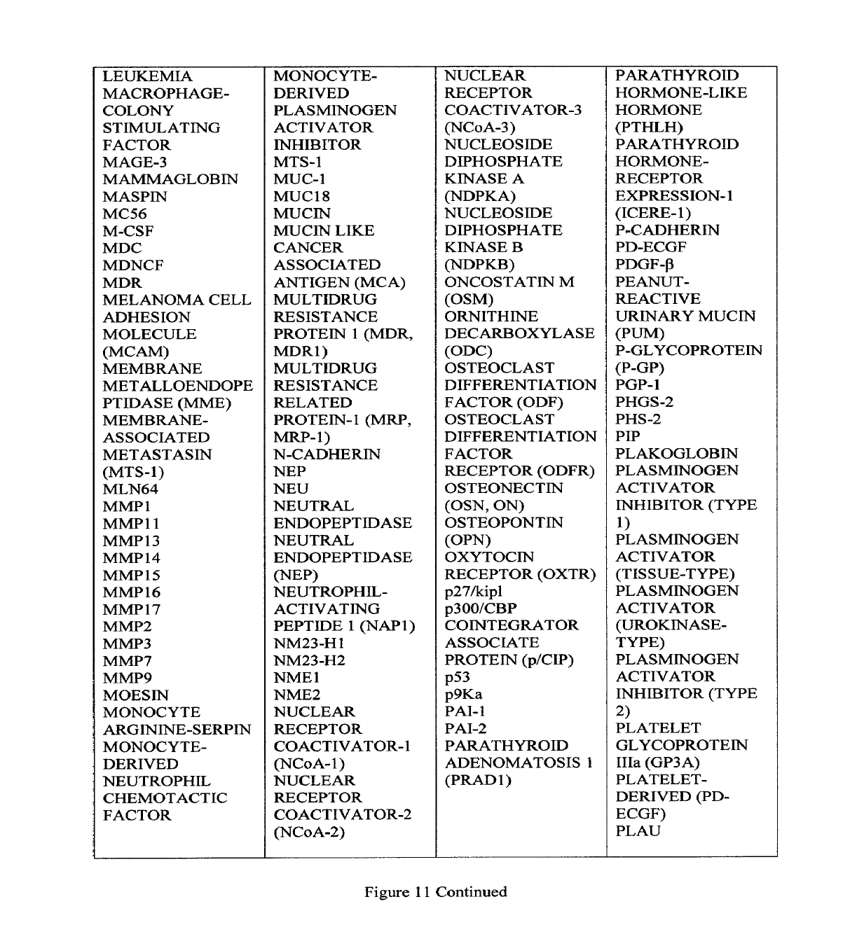

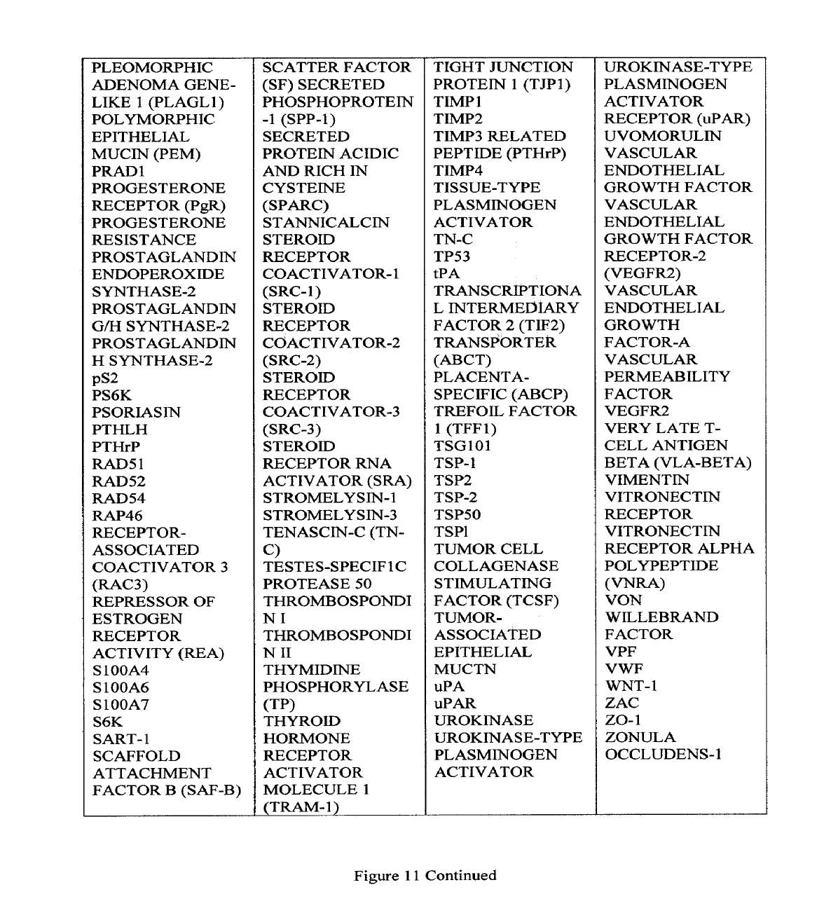

Preferably, the binding moieties selectively bind cell surface markers for cells of interest (e.g., cancer cells). Examples of markers that binding moieties may bind are those in Table 1 or any other marker described herein. More specific examples of binding moieties are antibodies such as anti-CD71, anti-CD235a, anti-CD36, anti-carbohydrates, anti-selectin, anti-CD45, anti-GPA, anti-antigen-i, anti-EpCAM, anti-E-cadherin, anti-Muc-1, or any antibody to a marker shown in FIG. 11. In particular, an antibody that specifically binds EpCAM, EGFR, or cytokeratin is contemplated. EpCAM may also be referred to as any of the following: GA733-2, EGP, GP40, EPG2, KSA, 17-1A, CO17-1A, esa, TACSTD1, CD326, M4S1, MIC18, MK-1, TROP1, and hEGP-2.

The one or more binding moieties can be attached to the enrichment device directly or indirectly. In some instances, the binding moieties (or a subset thereof) are attached to the device via a linker or more preferably a cleavable linker.

Linkers can be of different lengths and different structures, as is known in the art; see, generally, Hermanson, G. T., “Bioconjugate Techniques”, Academic Press: New York, 1996; and “Chemistry of Protein Conjugation and Cross-linking” by S. S. Wong, CRC Press, 1993, and U.S. Pat. No. 7,138,504 each of which are incorporated herein. Linking groups can have a range of structures, substituents and substitution patterns. They can, for example be derivitized with nitrogen, oxygen and/or sulfur containing groups which are pendent from, or integral to, the linker group backbone. Examples include, polyethers, polyacids (polyacrylic acid, polylactic acid), polyols (e.g., glycerol), polyamines (e.g., spermine, spermidine) and molecules having more than one nitrogen, oxygen and/or sulfur moiety (e.g., 1,3-diamino-2-propanol, taurine). See, for example, Sandler et al. Organic Functional Group Preparations 2nd Ed., Academic Press, Inc. San Diego 1983. A wide range of mono-, di- and bis-functionalized poly(ethyleneglycol) molecules are commercially available. See, for example, 1997-1998 Catalog, Shearwater Polymers, Inc., Huntsville, Ala. Additionally, those of skill in the art have available a great number of easily practiced, useful modification strategies within their synthetic arsenal. See, for example, Harris, Rev. Macromol. Chem. Phys., C25(3), 325-373 (1985); Zalipsky et al., Eur. Polym. J., 19(12), 1177-1183 (1983); U.S. Pat. No. 5,122,614, issued Jun. 16, 1992 to Zalipsky; U.S. Pat. No. 5,650,234, issued to Dolence et al. Jul. 22, 1997, and references therein.

A wide variety of linking chemistries are available for linking molecules to a wide variety of solid or semi-solid particle support elements. It is expected that one of skill can easily select appropriate chemistries, depending on the intended application. A linker can attach to a solid substrate through any of a variety of chemical bonds. For example, a linker can be optionally attached to a solid substrate using carbon-carbon bonds, for example via substrates having (poly)trifluorochloroethylene surfaces, or siloxane bonds (using, for example, glass or silicon oxide as the solid substrate). Siloxane bonds with the surface of the substrate are formed via reactions of derivatization reagents bearing trichlorosilyl or trialkoxysilyl groups. The particular linking group is selected based upon, e.g., its hydrophilic/hydrophobic properties where presentation of an attached polymer in solution is desirable. Groups which are suitable for attachment to a linking group include amine, hydroxyl, thiol, carboxylic acid, ester, amide, isocyanate and isothiocyanate. Other derivatizing groups include aminoalkyltrialkoxysilanes, hydroxyalkyltrialkoxysilanes, polyethyleneglycols, polyethyleneimine, polyacrylamide, polyvinylalcohol and combinations thereof. The reactive groups on a number of siloxane functionalizing reagents can be converted to other useful functional groups using methods known in the art. See, for example, Leyden et al., Symposium on Silylated Surfaces, Gordon & Breach 1980; Arkles, Chemtech 7, 766 (1977); and Plueddemann, Silane Coupling Reagents, Plenum, N. Y., 1982. Additional starting materials and reaction schemes will be apparent to those of skill in the art (U.S. Pat. No. 6,632,655).

Aptamers, affibodies or other linkers that exhibit a high affinity for the Fc portion of certain antibodies may be used to attach antibodies or antibody fragments to a solid object (e.g., U.S. Pat. No. 5,831,012).

A variety of cleavable linkers, including acid cleavable linkers, light or “photo” cleavable linkers and the like are known in the art Immobilization of assay components in an array is typically be via a cleavable linker group, e.g., a photolabile, acid or base labile linker group. Accordingly, a cell can be released from the device and/or the array of obstacles, for example, by exposure to a releasing agent such as light, acid, base or the like prior to flowing the cell to an output means. Typically, linking groups are used to attach polymers or other assay components during the synthesis of the device. Thus, linkers can operate well under organic and/or aqueous conditions, but cleave readily under specific cleavage conditions. The linker can, optionally, be provided with a spacer having active cleavable sites. Linking groups which facilitate polymer synthesis on solid supports and which provide other advantageous properties for biological assays are known. In some embodiments, the linker provides for a cleavable function by way of, for example, exposure to an acid or base. Additionally, the linkers optionally have an active site on one end opposite the attachment of the linker to a solid substrate in the array. The active sites are optionally protected during polymer synthesis using protecting groups. Among a wide variety of protecting groups which are useful are nitroveratryl (NVOC) α-methylnitroveratryl (Menvoc), allyloxycarbonyl (ALLOC), fluorenylnethoxycarbonyl (FMOC), α-methylnitro-piperonyloxycarbonyl (MeNPOC), —NH-FMOC groups, t-butyl esters, t-butyl ethers, and the like. Various exemplary protecting groups are described in, for example, Atherton et al., (1989) Solid Phase Peptide Synthesis, IRL Press, and Greene, et al. (1991) Protective Groups In Organic Chemistry, 2nd Ed., John Wiley & Sons, New York, N.Y.

In one aspect, coupling chemistries for coupling materials to the particles of the invention can be light-controllable, i.e., utilize photo-reactive chemistries. The use of photo-reactive chemistries and masking strategies to activate coupling of molecules to substrates, as well as other photo-reactive chemistries is generally known (e.g., for coupling bio-polymers to solid phase materials). The use of photo-cleavable protecting groups and photo-masking permits type switching of fixed array members, i.e., by altering the presence of substrates present on a device (i.e., in response to light) (U.S. Pat. No. 6,632,655).

In some embodiments, the cleavable linker comprises at least one of biotin/avidin, biotin/streptavidin, biotin/neutravidin, Ig-protein A, a photo-labile linker, acid or base labile linker group, an aptamer, an affibody or other linkers that exhibit a high affinity for the Fc portion of certain antibodies may be used to attach antibodies or antibody fragments to a solid object (e.g., U.S. Pat. No. 5,831,012).

Preferably, an enrichment device herein is covered with cleavable linkers comprising Neutravidin, avidin, or streptavidin protein. The cleavable linker can be cleaved by a DNase. In one example an anti-Ep-CAM antibody such as the following: biotin-polynucleotide-anti-EpCAM moiety is attached to the enrichment device which is covered with avidin.

Surfaces of the microfluidic device, including surfaces of an array of obstacles, a lid, a port, or some combination thereof, can be coated, (e.g. directly or indirectly linked) or coupled to at least one or two or more binding moieties. In some embodiments, combinations of two or more of such agents are immobilized upon the surfaces of the microfluidic device as a mixture of two or more entities or can be added serially. The surfaces of the microfluidic device can be treated with one or more blocking agents. For example, the surfaces of the microfluidic device can be treated with excess Ficoll or any other suitable blocking agent to reduce the retention of particles that lead to background signal when detecting one or more rare cells that can be retained by the microfluidic device.

Size Plus Affinity

In some instances a device herein is configured to retain cells of interest based on both size and affinity.

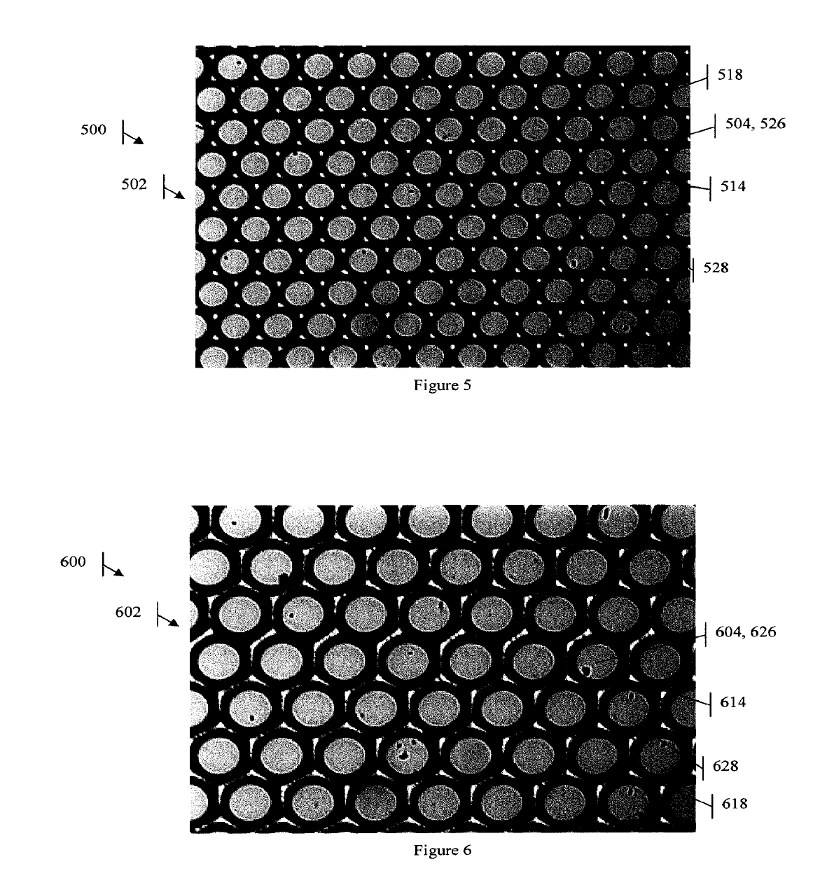

The device can comprise obstacles that are arranged uniformly or non-uniformly. One example of a uniform array is one where obstacles are configured such that each subsequent row in the array is offset by ½ the period of the previous row. (See FIGS. 4 and 5) Such arrays comprise a uniform gap size between all obstacles.

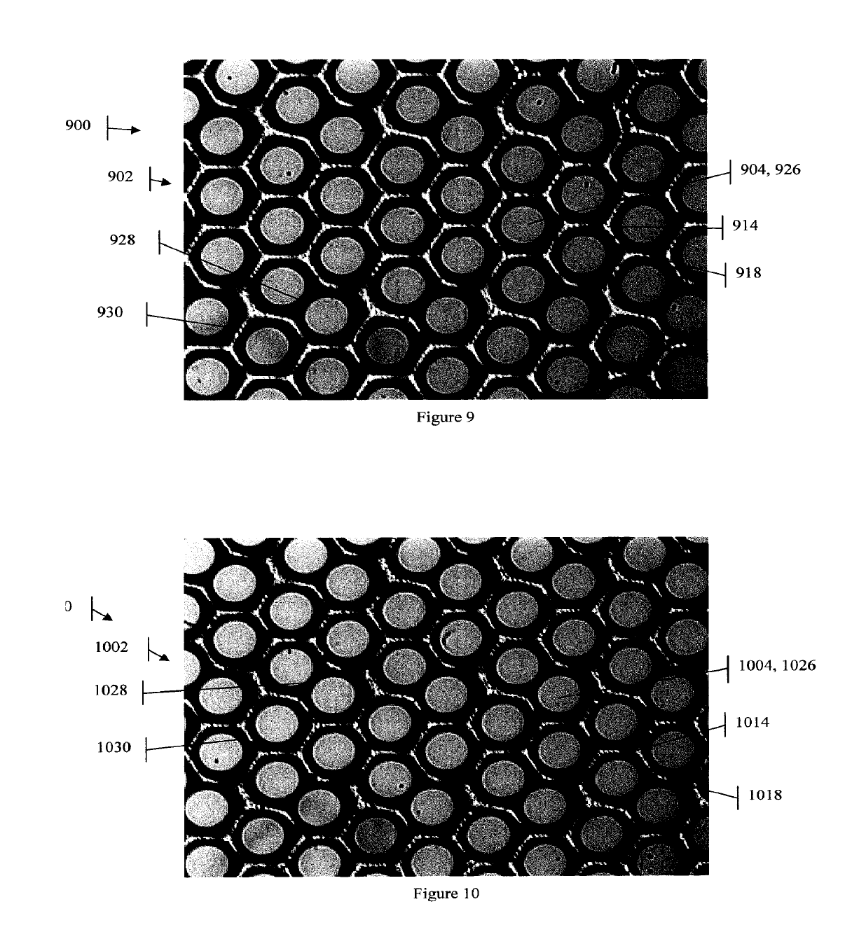

In some instances, a uniform array like the one described above comprises a subset of obstacles that are at an offset, such that they form a restricted gap with at least one obstacle. A restricted gap is one that is smaller than the average gap between all obstacles in an array. Such subset of obstacles can be distributed throughout the array in a uniform or non-uniform pattern. FIGS. 9-10 illustrate an array comprising a restricted gap at a uniform distribution. The number of restricted gaps can be up to 0.5%, 1%, 5%, 10%, 25%, or 40% of the total number of gaps between adjacent obstacles.

The enrichment devices herein are preferably made from a polymeric material, such as plastic.

The enrichment devices described herein can also include a lid that is optionally detachable, optically transparent, clear, or optically opaque. Moreover, the base layer of the device and the array of obstacles may also be optically transparent. This allows for optical detection means positioned adjacent to or above said array of obstacles to analyze cells retained within said array.

Use of a clear lid can allow visualization of detectable moieties bound to cells in the device.

Lids of said microfluidic device can be sealed to said device or removable. When cells are to be cultured following capture in a device, the lid can be removed prior to culturing cells in the device or following removal of target cells from the device using methods described elsewhere herein. The lid may be made from plastic, tape, glass or any other conventional material.

The device may also comprise a seal. A seal may be composed of at least one of an adhesive, a latch, or a heat-formed connection. A seal may be utilized for subsequent capturing of the cells or analysis or enumeration/visualization of the cells in the device.

Thus, preferably a device has a detachable, transparent lid, a seal, and an optically transparent base layer and array of obstacles.

The enrichment devices herein can further comprise one or more of the following features: a) an array of obstacles functionalized with binding moieties, wherein said array of obstacles comprises between 20 and 20,000 rows and between 10 and 1,000 columns of obstacles; b) an array of obstacles functionalized with binding moieties, wherein said array ob obstacles comprises at least 1000 obstacles; c) an array of obstacles functionalized with binding moieties that is adapted to process at least 0.5, 1, 1.5, 5, 10, 25, 500, or 1000 mL/hour of sample; d) an array of obstacles functionalized with binding moieties, wherein the binding moieties comprise two different binding moieties; e) an array of obstacles functionalized with binding moieties, wherein at least 50% of the surface area of the microfluidic device contacting the sample is functionalized with binding moieties; f) an array of obstacles functionalized with binding moieties, wherein the amount of surface area of the microfluidic device contacting the sample is at least 30, 50, 75, 100, 250, or 500 mm²; g) an array of obstacles enclosed by a chamber, wherein the chamber can hold at least 2, 5, 10, 25, 50, or 100 μL, of fluid; h) an array of obstacles enclosed in a chamber, wherein at least 5%, 10%, 25%, 35%, 50%, or 65% of the interior volume of said chamber is occupied by said obstacles; i) an array of obstacles, wherein said array of obstacles comprises a first array of obstacles fluidly coupled to a second array of obstacles, and further wherein said first array of obstacles has a restricted gap dispersed in a uniform pattern and said second array of obstacles has a uniform pattern of obstacles and no restricted gap; j) an array of obstacle functionalized with one or more binding moieties, wherein the array of obstacles are fixed to the microfluidic device; or k) an array of obstacles functionalized with one or more binding moieties, a lid, and a port. The binding moieties can be anti-EpCAM or anti-EGFR.

In some instances, an enrichment device herein comprises two or more of the previously described features. For example, the microfluidic device can comprise features a) and g) or a) and b) and h).

The enrichment devices herein can also include one or more inlet ports and one or more outlet ports. A port is any region used for delivering fluid to or removing fluid from an enrichment module, such as an array of obstacles. Inlets or inlet ports refer to modules or opening that are used for delivering fluid to an enrichment module. Outlets or outlet ports refer to modules or opening that are used for removing fluid from an enrichment module.

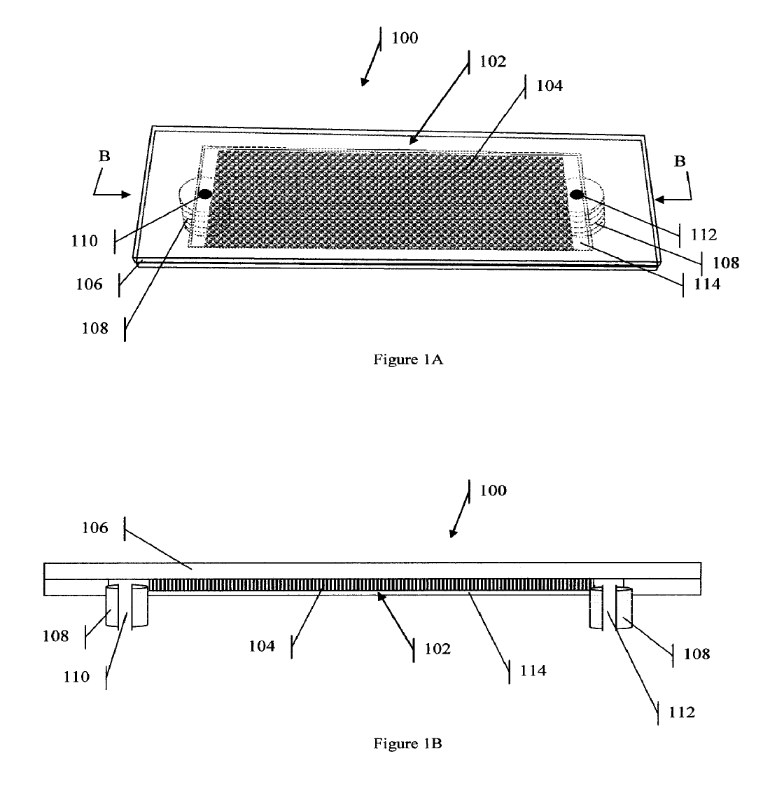

For example, FIGS. 1A and 1B depict a microfluidic device having a lid and removable threaded screw ports attached to the inlet and to the outlet. FIG. 1B Depicts a cross-sectional view of the microfluidic device of FIG. 1A having a lid and removable screw ports, cut along line B-B of FIG. 1A.

Application of the sample to the a microfluidic device comprising a chamber with an array of obstacles for enriching one or more rare cells from a fluid sample comprising rare cells and non-rare cells may be accomplished with tubing connecting the chamber to a fluid sample source. The tubing may be any conventional material such as teflon, silicone or plastic.

Provided herein is a microfluidic device for enriching one or more rare cells from a fluid sample comprising rare cells and non-rare cells, the device comprising a chamber having a base layer, an array of obstacles arising from the base layer, a plurality of gaps between obstacles, an outlet, and an inlet. In some embodiments, the outlet comprises an inlet removable port, and wherein the inlet comprises an outlet removable port. In other embodiments, the inlet removable port connects to a sample reservoir. In other embodiments, the removable ports are break-away screws having a channel therethrough. An example of a removable port 108 is shown in FIG. 1A and in FIG. 1B.

A port refers to an opening in the device through which a fluid sample or any other fluid can enter or exit the device. A port can be of any dimensions, but preferably is of a shape and size that allows a sample or the desired fluid or both to be dispensed into a chamber by pumping a fluid through a conduit (or tube, or tubing) or by means of a pipette, syringe, or other means of dispensing or transporting a sample.

An inlet can be a point of entrance for sample, solutions, buffers, or reagents into a fluidic chamber, such as the microfluidic device described herein. An inlet can be a port, or can be an opening in a conduit that leads, directly or indirectly, to a chamber of an automated system.

An outlet refers to an opening at which sample, sample components, reagents, liquids, or waste exit a fluidic chamber, such as the microfluidic device described herein. The sample components and reagents that leave a chamber can be waste, i.e., sample components that are not to be used further, or can be sample components or reagents to be recovered, such as, for example, reusable reagents or target cells to be further analyzed, manipulated, or captured. An outlet can be a port of a chamber such as the microfluidic device described herein, or an opening in a conduit that, directly or indirectly, leads from a chamber of an automated system.

In some embodiments, the device may comprise multiple inlets, multiple outlets, or a combination thereof associated with a single array of obstacles and fluid sample. In some embodiments, the device may comprise multiple inlets, multiple outlets, or a combination thereof associated with multiple arrays of obstacles for processing a single sample, or multiple samples or both in series or in parallel or both.

A conduit refers to a means for fluid to be transported from a container or vial to a chamber such as the microfluidic device described herein. In some embodiments, a conduit directly or indirectly engages a port in the microfluidic device described herein. A conduit can comprise any material that permits the passage of a fluid through it. Conduits can comprise tubing, such as, for example, rubber, Teflon, or Tygon tubing. Conduits can also be molded out of a polymer or plastic, or drilled, etched, or machined into a metal, glass or ceramic substrate. Conduits can thus be integral to structures such as, for example, a cartridge of the present invention. A conduit can be of any dimensions sufficient to flow the sample or the buffer or both through the microfluidic device described herein. A conduit is preferably enclosed (other than fluid entry and exit points), or can be open at its upper surface, as a canal-type conduit.

In some embodiments, the inlet means includes a well that will contain between about 1 mL and about 1.5 L of liquid. A well refers to a structure in the microfluidic device or connected to the inlet port of the microfluidic device for holding the sample or another liquid prior or subsequent to flowing through the microfluidic device. The well may be a vial, or another means for holding the sample or other reagents such as buffer.

Microfluidic devices and methods for enrichment of rare cells based on size, affinity, deformability, and shape are described in co-pending U.S. application Ser. No. 11/322,791.

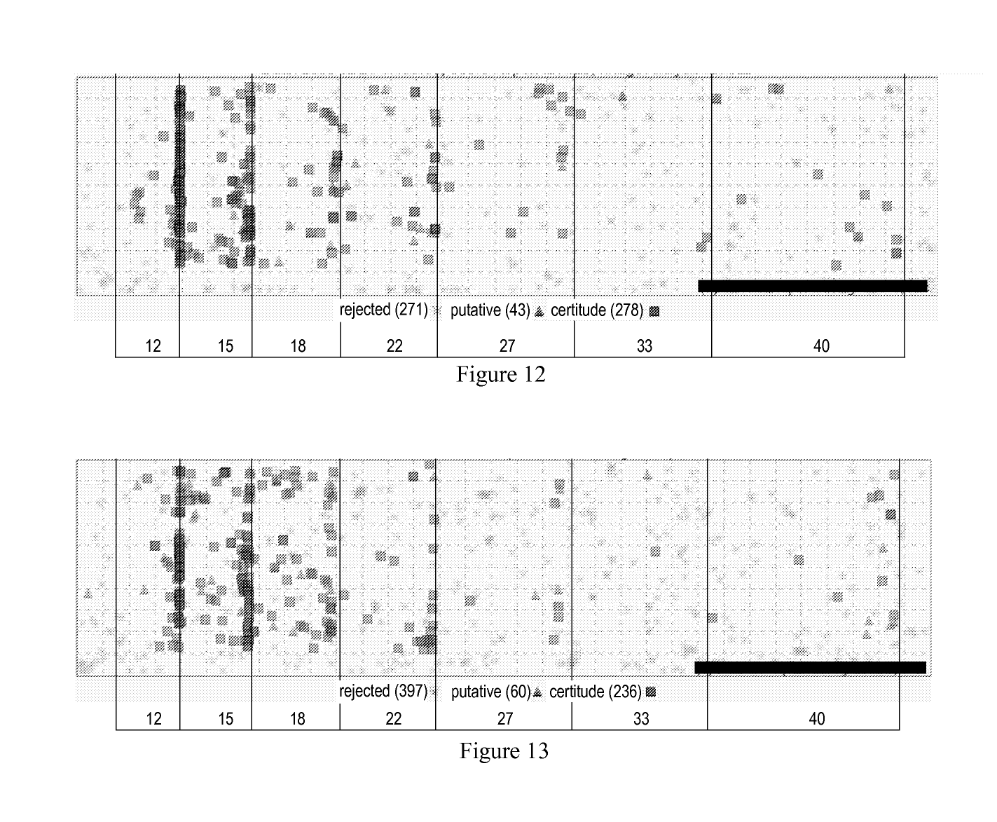

In some instances, enrichment devices contemplated herein perform both size and affinity separation. Such devices can comprise two or more subarrays, each of which is fluidly coupled to the others in series. FIG. 12 illustrates an example of such an array. The first subarray, which is located upstream of a second subarray, has an average gap length between its obstacles that is bigger than the average gap length of the second subarray. A third subarray located downstream of the second subarray, has an average gap length between its obstacles that is smaller than the second subarray. Such an array can be composed of at least 2, 3, 4, 5, 6, 7, 8, 9, 10 or 20 subarrays.

In some instances, the average gap length in a first subarray upstream to a second subarray is greater than 35, 25, 20, or 15 microns or up to 60, 50, 40, 30, 20 or microns. The average gap length of a second subarray downstream from the first subarray is greater than 25, 20, 15, or 10 microns or up to 12, 15, 18, or 22 microns. The average gap length of a third subarray fluidly coupled downstream to the second array is greater than 5, 10 or 12 or up to 17, 15, or 13 microns.

The enrichment device above can be covered by one or more different binding moieties that. The binding moieties can be a protein, nucleic acid, or small molecule associated with a marker shown in FIG. 11. The binding moieties preferably bind a cell surface marker in cells of interest. In some instances, an array comprises one or more binding moieties that selectively binds cells of interest and one or more binding moieties that selectively binds non-cells of interest. Such first and second binding moieties are located in different regions in the array. One skilled in the arts would know which binding moieties to choose from the markers shown in FIG. 11 based on cells of interest.

For example, each of the subarrays can be functionalized with the same or a different pattern or binding moieties. In an array comprising multiple distinct regions of moieties, a first region can comprise a set of one or more binding moieties and a second region can comprise a different set of one or more binding moieties. The first set and the second set of one or more binding moieties can include anti-EpCAM, anti-EGFR, anti-cytokeratin, anti-LAR, or any binding moiety to any marker described herein. The first region can be distinct from the second region or the same.

Any of the devices herein can be configured such that in any one or more subarrays, at least 5, 10, or 20% of the cells are captured within the first 10, 20, 30, 40 or 50 rows of such subarray.

Since the enrichment device herein can retain and sort cells based on size and cell surface markers/affinity, such device can be used to profile an individual's cell population or a subset of the cell population (e.g., those cells larger than 6 microns in diameter).

For example, a first cell profile can comprise a number of cells of a first type retained in a first subarray of the microfluidic device within a first region having a first set of binding moieties and a second type of cell can be retained in a second or third subarray at a region having a second set of binding moieties. For example, CTCs undergoing apoptosis may be captured in a subarray downstream from non-apoptotic CTCs that might be larger in size and captured upstream. Similarly, circulating tumor stem cells may be captured in a different region of a first array than tumor non-stem cells based on their unique cell surface markers.

Thus, the present invention contemplates diagnosing, prognosing, or theranosing a condition in a patient, by flowing a sample from the patient through an array of obstacles that performs both size and affinity sorting, such as the devices described herein, and using the cell profile of the patient to diagnose, prognose or select a treatment. For example, if most CTCs from a patient's blood sample are undergoing apoptosis and are normal than non-apoptotic CTCs, a patient may remain on an ongoing treatment regimen or may stop treatment altogether. On the other hand, if most of the cells captured from a patient's blood sample are circulating tumor stem cells, a more aggressive treatment regimen may be required.

Any of the enrichment devices herein may be configured to enrich at least one rare cell from a fluid sample from at least 10, 20, 25, or 50% of at least stage 1 cancer patients.

The rare cell(s) enriched can be ciruclating epithelial cells or CTCs. The fluid sample can be a blood sample of up to 1.5 L, or up to 1 L, or up to 500 mL, or up to 100 mL, or up to 50 mL, or up to 10 mL.

The stage 1 cancer patients may be stage 1 lung cancer patients, stage 1 breast cancer patients, stage 1 colon cancer patients, stage 1 prostate cancer patients, or stage 1 ovarian cancer patients.

In some instances, the device is configured to enrich at least 1 rare cell from at least 10, 20, 25, or 50% of at least stage 2 cancer patients as described above.

Preferably, the device enriches the rare cell(s) without mechanically damaging them due to the low shear experienced by the rare cells. The low shear can be described as having a Reynolds number for fluid flow through the microfluidic device less than about 0.01, or between about 0.01 and about 0.0005, or less than about 0.0005. Mechanically damaging the rare cells can include rupturing the cells or causing the rare cells to undergo apoptosis.

The device can be configured to not comprise magnetic beads.

The device would comprise an array of obstacles forms a network of gaps between adjacent obstacles, and further wherein the gaps between adjacent obstacles are between 1 and 300 microns in length. The obstacles are covered by one or more binding moieties include anti-EpCAM antibodies.

Thus, methods of the invention include diagnosing, theranosing, or prognosing cancer in a patient comprising the following steps: obtaining a sample from said patient, flowing said sample through a microfluidic device adapted for retaining one or more rare cells in at least 5, 10, 20, 25, or 50% of patients having at least stage 1 of said cancer, and making a diagnosis, theranosis, or prognosis based on retained cells. The one or more rare cells are not mechanically damaged by flowing said sample through the microfluidic device. The one or more rare cells can be circulating tumor cells or epithelial cells. The microfluidic device can comprise one or more binding moieties and/or an array of obstacles. The array of obstacles can form a network of gaps between adjacent obstacles. The gaps between adjacent obstacles can be between 1 and 300 microns in length. The one or more binding moieties can include anti-EpCAM.

Cell Fragments

It is understood that a device that selectively binds rare cells based on cell surface markers also binds fragments of such rare cells with the cell surface marker.

Thus the present invention contemplates diagnosing a condition such as cancer in a patient by quantitating total cells and cell fragments enriched using the devices herein. In some instances, the cells and cell fragments are labeled with a fluorescent label and total fluorescent is determined. Analysis using this system is made using a fluorescent microscope. In some instances the cells and cell fragments are labeled with an immunochemical stain and total volume of cells and number of cells and cell fragments is determined using a bright field microscope.

In either method, the stains selectively bind a cancer marker, such as, e.g., cytokeratin, EGFR, EpCAM, cadherin, mucin, or LAR. In some instances, an enriched sample is stained with a cytokeratin colorimetric or luminescent stain, or more preferably a cytokeratin 19 stain, and analyzed under a bright field microscope. Examples of stains that can be used herein include a pan-cytokeratin antibody, a biotinylated secondary antibody, an avidin-biotinylated horseradish peroxidase complex, and diaminobenzidine tetrahydrochloride. The pan-cytokeratin antibody can be a mixture of monoclonal antibodies, for example AE1/AE3 antibodies.

Enumerating of stained cells and cell fragments can comprise measuring total amount of stained area or measuring total intensity of stained area. Such measurements can be made using a processor. The processor enumerates the one or more enriched circulating tumor cells and their fragments using an image of the enriched circulating tumor cells taken by a bright field microscope.

Prior to staining, the cells are enriched using any of the devices herein that performs size and/or affinity enrichment using a two-dimensional array of obstacles. In some instances, the obstacles are functionalized with at least one set of binding moieties.

In some instances, staining includes using an indicator for determining a tissue of origin for the one or more enriched circulating tumor cells. Such staining can include an indicator for determining efficacy of a cancer therapeutic agent. Such staining can include a fluorescent dye.

The present invention also relates to kits comprising a microfluidic device for enriching rare cells and at least one immunochemical stain (such as those that selectively bind cytokeratin) that is visualized using a bright field microscope that selectively binds enriched rare cells or fragments thereof.

Sample Flow and Incubation

Enriching rare particles and cells involves flowing a sample through an enrichment device, e.g., such as any of the ones described herein comprising an array of obstacles. The sample flow rate can be reduced once the enrichment device is loaded with sample. The sample flow rate can be reduced to zero and the sample can be allowed to incubate in the enrichment device. Moreover, sample outlet can be fluidly coupled to a sample inlet such that a sample may circulate multiple times through the device, allowing the rare cells more opportunities to selectively bind or be selectively captured by the device.

In some instances, only a portion of the sample is removed from the device and optionally allowed to further contact the device.

Thus, the enrichment methods herein comprise flowing a sample into a device as described herein; allowing the sample to remain in contact with a first array of obstacles in the device for a period of time; and optionally flowing at least a portion of the sample out.

The period of time that the sample is in contact with the array can be at least 0.5, 2,5, 10, 15, 30, 60, or 120 minutes. Alternatively, the step of allowing the sample to remain in contact with the first array of obstacles can comprise flowing the sample through the first array of obstacles at a reduced flow rate of up to 0.1 mL/hr. An array can be configured to be large enough to still remain in contact with the first array of obstacles for more than 0.5, 2, 5, 10, 15, 30, 60, or 120 minutes, even at a reduced flow rate.

While the sample can flow through the first array of obstacles at a steady flow rate, it can also be configured to flow through the first array of obstacles in a pulsed flow pattern (flow, rest, flow, rest, etc.). In some embodiments, the pulsed flow pattern comprises a flow time of about 5 seconds to about 5 minutes, or about 10 seconds, about 30 seconds, about 45 seconds, about 1 minute, about 1.5 minutes, about 2 minutes, about 3 minutes, or about 4 minutes, and a rest time of about 1 second to about 1 minute, or about 3 seconds, about 5 seconds, about 10 seconds, about 30 seconds, or about 45 seconds. The rest time can be identical to the flow time, shorter, or longer. The flow time and the rest time can be alternated in a regular or irregular pattern.

In other embodiments comprising, as described herein, a flow generator, an inlet, an inlet port, an outlet, and an outlet port or some combination of these elements, each of said obstacles has a surface providing binding moiety, said binding moiety attached to said surface of said structure via a cleavable linker and capable of specifically binding said rare cells.

The flow of the sample through the device also provides opportunities for the rare cells to bind with binding moieties on the array, on the lid, if present, and on the base layer surface. The obstacles in the devices herein are arranged to allow the rare cells (e.g. CTCs) to roll through the microfluidic channels and maximize contact with the device surface having binding moieties. Binding moieties can be selective for any cell type, such as, for example, trophoblasts, circulating tumor cells, epithelial cells, circulating tumor cells, cancer cells or cancer stem cells. The microfluidic devices may have antibodies specific for target cells or non-target cells immobilized within the microfluidic devices. Various antibodies are contemplated and discussed herein.

Binding moieties (e.g. Abs) may be attached to the base (e.g. base layer), the facing surface (e.g. lid), the obstacles, and the sidewalls of the collection region in the device. It has been determined that flow of liquid containing cells or other biomolecules through even a confined lumen results in the cells being primarily present in the central flow stream region where flow shear is the least. As a result, capture upon sidewalls that carry binding moieties is sparse in comparison to the capture upon surfaces in the immediate regions where the transverse obstacles have disrupted streamlined flow. In these regions, binding moieties can assume their native 3-dimensional configurations as a result of proper coupling and can be effective for binding rare cells.

A bodily fluid, such as a blood or urine sample or other sample described herein, or some other pretreated liquid containing or being suspected of containing the target cell population, is caused to flow through gaps between the obstacles (or the collection region), as by being discharged carefully from a standard syringe pump or by means of another flow generator and method of flow generation described herein into an inlet passageway leading to the inlet for such a microchannel device or drawn by a vacuum pump, peristaltic pump, or the like therethrough from a sample reservoir provided by a relatively large diameter inlet passageway which serves as well to hold the desired volume of sample for a test. The opening may contain a fitting (or inlet port or outlet port having a bore therethrough) for mating with tubing connected to such a flow generator, a reservoir, or pump described herein when such is used. The pump or flow generator may be operated to effect a flow of about 0.5-10 μL/min. through the apparatus. Depending upon the bodily fluid, or other cell-containing liquid that is to be treated and/or analyzed, a pretreatment step may be used to reduce its volume and/or to deplete it of undesired biomolecules, as is known in this art.

Provided herein is a microfluidic device for enriching one or more rare cells from a fluid sample comprising rare cells and non-rare cells, the device comprising a chamber having a base layer, an array of obstacles arising from the base layer, and a plurality of gaps between obstacles, wherein the device is adapted and configured to flow the fluid sample through the chamber at a rate of, for example, about 0.1 mL/hr, about 1 mL/hr, between about 0.5 mL/hr and about 2.0 mL/hr, between about 5 μL/min and about 50 μL/min, about 10 mL/hr, about 30 mL/hr, at least 0.1 mL/hr, and at most 30 mL/hr. When referring to flow rate of either sample or buffer or both or any liquid through the device, “about” refers to variations in flow rate of 0.01 mL/hr to 0.05 mL/hr for slower flow rates, or of 1 mL/hr to 5 mL/hr for faster flow rates.

Provided herein is a microfluidic device adapted and configured to flow buffer through the chamber at a rate, for example, of about 0.5 mL/hr, between about 1 mL/hr and about 20 mL/hr, between about 10 μL/min and 200 μL/min, about 10 mL/hr, about 30 mL/hr, at least 0.5 mL/hr, and at most 30 mL/hr. When referring to flow rate of either sample or buffer or both or any liquid through the device, “about” refers to variations in flow rate of 0.01 mL/hr to 0.05 mL/hr for slower flow rates, or of 1 mL/hr to 5 mL/hr for faster flow rates.

The methods of the invention can comprise flowing a volumetric rate of sample through a microfluidic device and retaining one or more cells. The volumetric rate of sample can be between about 1 and 2,000 mL/hr, between about 5 and 1,500 mL/hr, between about 20 and 1000 mL/hr, or between about 50 and 500 mL/hour. The microfluidic device can comprise no more than one microfluidic device. In some embodiments of the invention, the microfluidic device can comprise an array of obstacles, one or more binding moieties, or any combination thereof.

The area occupied by an obstacle can be up to, about, or less than 5%, 10%, 20%, 30%, 40%, 70% of the area inside the boundary defining the microfluidic device. A sample can be driven through the microfluidic device from an inlet port to an outlet port using hydrodynamic force. In some embodiments of the invention, the hydrodynamic force can be pressure.

The sample is flowed through the device in such a way to reduce all turbulences or eddies. The Reynolds numbers for a blood sample flowing through the device, for example, can be less than about 0.0100, or between about 0.0100 and about 0.0005, or at least about 0.0005.

In some embodiments of the device having a first gap and a second gap as described herein, the device is adapted and configured to flow the fluid sample and/or buffer through the chamber at a rates described herein.

In other embodiments of the device having a first gap and a second gap as described herein and at least one of a fluid sample flow rate as described herein, and a buffer flow rate as described herein, the device is adapted and configured to flow the buffer and/or the liquid sample through the chamber at a steady flow rate, in a pulsed flow pattern as previously described, or a combination thereof.

In yet other embodiments the microfluidic device is adapted and configured to flow said liquid sample and said buffer in at least one of the same direction, differing directions, and opposite directions.

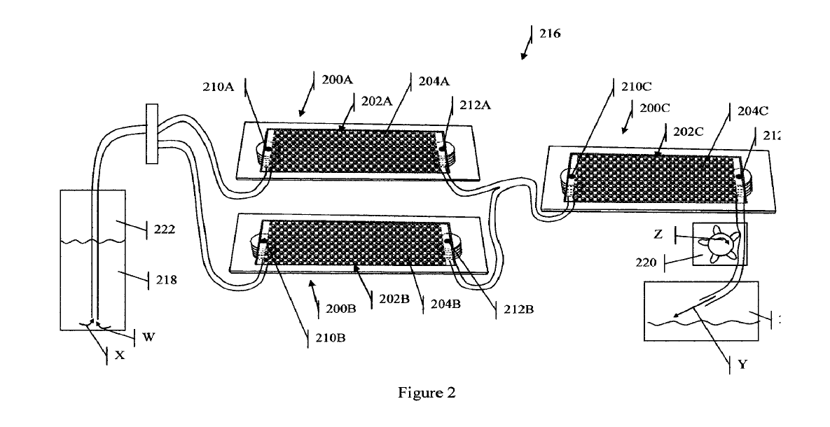

Provided herein is a system for enriching one or more rare cells from a fluid sample comprising rare cells and non-rare cells comprising a first microfluidic device, the first device comprising a chamber having a base layer, an array of obstacles arising from the base layer, and a plurality of gaps between obstacles, and a second microfluidic device, the second device comprising a chamber having a base layer, an array of obstacles arising from the base layer, and a plurality of gaps between obstacles, wherein the first and the second microfluidic device are adapted and configured to flow the liquid sample through said devices in parallel or in series, or in a combination thereof. An example of such a system 216 is shown in FIG. 2 and is previously described.

In one system embodiment, the first microfluidic device comprises an inlet port and a first outlet port. In another system embodiment, the first microfluidic device comprises a second outlet port. In another system embodiment, the first outlet port connects to an inlet port of the second microfluidic device.

The inlets and outlets may be connected to reprocess a sample through the same array, to additionally process a sample through a new array, wherein the array may be provide size or affinity capture or a combination thereof, and may capture by the same manner or by using a new detection means or antibody or binding moiety.

Internal Standards and Capture Efficiency

The devices and methods herein also permit the determination if a subject has a critical concentration of rare cells. The method can include generating a sample test solution comprising a standard that mimics rare cells. The standard can be discrete particles comprising tumor cell antigens. The standard can be applied to a plurality capture elements and the number of discrete particles recovered can be used to determine capture efficiency. The capture efficiency can be used in assays for determining if a subject has the critical concentration of circulating tumor cells. Results of determining if a subject has a critical concentration of rare cells can be reported to the subject. The rare cells can be circulating tumor cells.

The method for determining if a subject has a critical concentration of circulating tumor cells or rare cells can comprise generating a sample test solution by adding a known number of discrete particles to a sample obtained from the subject, where the discrete particles can comprise a circulating tumor cell antigen or rare cell antigen. The method can further comprise contacting the sample test solution with a plurality of capture elements comprising a binding moiety that binds specifically to the circulating tumor cell antigen or rare cell antigen. Determining the number of discrete particles captured by the plurality of capture elements and determining the number of circulating tumor cells or rare cells captured by the plurality of capture elements can be used to determine if the subject has a critical concentration of circulating tumor cells or rare cells. The results of determining if the subject has a critical concentration of circulating tumor cells can be reported to the subject, used to diagnose, prognose, or theranose a condition in the subject, or used to select a patient for a clinical trial.

The discrete particles can be any particle that mimics circulating tumor cells or rare cells. The discrete particles can be agarose beads or dendrimers. The discrete particle can have an average size that can be 0.5, 1, 2, 4, 5, or 10 microns larger or smaller than the average size of the circulating tumor cell or rare cell that can be captured by the plurality of capture elements. The discrete particle can be labeled with a first dye and the circulating tumor cells or rare cells can be labeled with a second dye. The first dye and the second cell can have light absorption wave lengths or fluorescent light emission wavelengths that are separated by at least 5, 10, 20, 40, 50, 75, or 100 nm. The discrete particles can be functionalized with a circulating tumor cell antigen, e.g. EpCAM or EGFR.

The determining if the subject has a critical concentration of circulating tumor cells or rare cells can be based on the capture efficiency determined by the number of discrete particles captured and the number of discrete particles added to the sample, the expected number of circulating tumor cells captured by the plurality capture elements for a subject having the critical concentration, the number of circulating tumor cells captured by the plurality of capture elements, and the total volume of the samples contacted with the plurality of capture elements.

The plurality of capture elements can comprise an array of obstacles functionalized with said one or more binding moieties. The array of obstacles can be fixed to a microfluidic device and/or the array of obstacles can form a network of gaps between adjacent obstacles that are between 5 and 300 microns in length.

The absence of circulating tumor cells or rare cells captured by the plurality of capture elements can indicate that the likelihood that the subject has the critical concentration of circulating tumor cells or rare cells can be less than a diagnostic level.

The methods and devices described herein are used to determine a likelihood if a subject has a critical concentration of rare cells. The term “critical concentration” refers to a minimum concentration of rare cells (e.g., circulating tumor cells, tumor cells, total tumor cells, viable tumor cells, or tumor stem cells) in a subject's circulation that warrants follow-up medical intervention, e.g., follow-up assays (e.g., biopsies, in vivo imaging analysis, blood tests etc.) or cancer therapy (e.g., chemotherapy, radiotherapy, surgery, or combinations thereof). In some embodiments, the critical concentration of CTCs or rare cells can be between about 1 to 10, 1 to 20, 20 to 40, or 40 to 100 cells per 10 ml of blood.

In some embodiments, the assays described herein detect the presence of a critical concentration of rare cells in a subject with a likelihood that can be equal to or less than a diagnostic risk level. The diagnostic risk level refers to the probability that a rare cell detection assay fails to detect a single rare cell in a subject having a rare cell concentration equal to at least the critical concentration. Accordingly, the overall probability that a subject has the critical concentration, can be determined based on: (1) the difference between the number of detected rare cells for a given volume of sample from the subject that was assayed, the expected number of rare cells for the same sample volume from a subject having the critical concentration; and (2) a capture efficiency for a plurality of capture elements (e.g., magnetic beads, posts, channels, or other structures) used to detect the rare cells. The maximum acceptable diagnostic risk level can be pre-defined to be no greater than, e.g., 0.02, 0.01, 0.001, 0.0001, 0.00001, 0.000001, or 0.0000001.

In some embodiments, the rare cell detection assay can be designed to have a diagnostic risk level value according to the following equation (Eq. 1):

P=[1−(ΔCD×E)]^{V} [Eq. 1]

Where:

P: the probability that the subject has a critical concentration when the concentration of detected rare cells (D) is lower than would be expected for a subject having the critical concentration (C) based on the total volume of samples analyzed from the subject, and the capture efficiency of the rare cell detection method;

ΔCD: the difference between the expected concentration of rare cells for a subject having the critical concentration (cells/mL) and the concentration of detected rare cells (cells/mL) in the volume from one or more samples V from the subject, or C-D;

E: the capture efficiency of the rare cell detection method;

V: the total volume of the sample analyzed from the subject (mL)

The following exemplary embodiment illustrates a CTC detection assay designed a pre-defined diagnostic risk level:

Assume:

(1) the desired (i.e., pre-defined) diagnostic risk level of the assay is 0.01 (P), i.e., a probability of 1/100 or less that the subject has the critical CTC even if fewer cells are detected than in one or more samples having a total volume (V);

(2) the critical CTC concentration is taken as 0.3 CTCs/ml of blood (C); and

(3) the target capture efficiency of the rare cell enrichment method (E) is 0.70 (i.e., on average, 70% rare cells cells in a given sample volume are captured by the enrichment method); and

(4) in this instance, 0 CTCs are detected in the total volume of one or more biological samples from the subject, i.e., D=0, and therefore ΔCD=0.3. Thus, according to Eq. 1:

(i) 0.01=[1−(0.3×0.7)]^{V}

(ii) 0.01=[1−0.21]^{V}

(iii) 0.01=0.79^{V}

Solving, (iii) for V by reiteration, V=19.5 ml. Thus, if 0 cells were detected in 19.5 ml of blood from a subject, the subject would still have a 1/100 chance of having a critical CTC concentration of 0.3 CTC/ml notwithstanding the failure to detect a single tumor cell, i.e., the odds that the assay failed to detect the minimum number of CTCs by pure chance is 1/100.

By extension, if a number of CTCs were detected in a total sample volume such that D=0.1 CTC/ml, then according to Eq. 1:

(i) 0.01=[1−((0.3−0.1)×0.7)]^{V}

(ii) 0.01=[1−0.14]^{V}

(iii) 0.01=0.86^{V}

Solving (iii) for V by reiteration, V=30.5 ml. Thus, in this case, where 315 cells are detected in 30.5 ml of blood from a subject, the subject would has a 1/100 chance of having a critical CTC concentration of 0.3 CTC/ml notwithstanding that based on the number of detected tumor cells, the concentration of CTCs is 0.1 CTC/ml, i.e., the odds that the assay failed to detect the minimum number of CTCs by pure chance is 1/100.

Accordingly, in some embodiments, the CTC analysis methods described herein utilize a minimum total sample volume based on a maximum acceptable diagnostic risk level, a critical CTC concentration, and a capture efficiency for the chosen enrichment method (e.g., enrichment using a microfluidics device as described herein).

In some embodiments, the CTC analysis methods described herein include determining a capture efficiency (E) of a specific rare cell enrichment method for a biological sample from a specific subject. Determining a sample-specific capture efficiency fulfills at least two objectives: first, it shows that the chosen rare cell enrichment method is performing adequately for a specific sample, and second, it allows the assay results to be normalized relative to sample-specific differences in capture efficiency, thereby increasing their accuracy and reliability vis-a-vis a diagnostic risk level. By way of illustration only, referring to Eq. 1, if for patient A:

V_{A}=20 ml; ΔCD_{A}=0.3 (i.e., 0 cells detected); and the subject sample-specific capture efficiency E_{A}=0.7, then the diagnostic risk level for subject A is P_{A}=[1−(0.3×0.7)]²⁰, i.e., P_{A}=0.0090≈0.01;

On the other hand, for patient B, having identical assay results/parameters except for a different subject sample-specific capture efficiency E_{B}=0.5, the diagnostic risk level

P_{B}=[1−(0.3×0.5)]²⁰, i.e., P_{B}=0.039≈0.04.

Thus, despite finding 0 tumor cells in both patient samples, Patient B's diagnostic risk level would be approximately four fold higher than patient A's diagnostic risk level due to the difference in sample-specific capture efficiencies.

Capture efficiency of an enrichment device herein may also be evaluated using bead or other particles. Beads or particles that are smaller than the smallest spacing between obstacles in an enrichment device or smaller than CTCs are functionalized as targets (e.g., CTCs or circulating tumor stem cells or epithelial cells). This allows them to specifically bind to the binding moieties on the array (e.g. anti-Ep-CAM antibodies). Beads used in this context can be configured to fluoresce at a wavelength different than any of the stains used to identify cells. If 100% of beads are captured by the device, one can assume that the device has a 100% capture efficiency. Similarly if only 90% of all beads/particles functionalized are captured, the capture efficiency of the device would be 90%.

If an array is designed to capture multiple targets or cells, beads that are distinctly shaped (e.g. shapes that can be easily differentiated from a cell and between bead specificities) can be used to distinguish capture specificity. Beads of this type can also be used to evaluate the flow patterns of the samples run over the array. Thus, by flowing beads through the array with the sample, one can determine binding efficiency (e.g., are beads interfering with some of the target binding sites).

The beads or particles described above can also be used to evaluate quality of reagents used. In this embodiment, another set of beads is added that are functionalized with targets for the stains used in cell capture (e.g., cytokeratin or gene regions such as BRAC1, or SNP regions of interest). The beads or particles used in this quality control process are larger than the largest gap size between two obstacles in an array, such that all of the beads are captured. The beads can also be smaller than the smallest gap between obstacles and further comprise antigens to the binding moieties on the device.

When staining is applying to the captured cells, the beads/particles will be stained as well. The beads are in sizes that are designed to get stuck at specific post spacings. Such that the bead is recognized by a fluorescent color for the bead (identifying a bead and not a cell), by the presence of the color the stain reagent the bead is specific for (showing that reagent is functional), and finally by the region of the chip that the bead was captured in (immobilization only by size). Since there is a known number of beads or particles combined with the sample, it is possible to standardize the number of rare cells captured and determine reagent efficiency based on stains from the control beads. If there are multiple unique target beads, each would be a different size from the other types of target beads.

Moreover, the invention herein contemplates the use of beads or particles to evaluate cell capture. Under this embodiment, beads are designed to mimic cell binding mechanisms well enough to provide predictive data on cell capture efficiency. Such beads “behave” like cells through the array of obstacles. For example, beads can be prepared using soft materials (e.g., agrose) or be large loosely structured chemical entities (e.g., dendrimers). Such soft materials allow the beads to morph their shapes much like cells do as they flow through an array of obstacles. Beads of this type can be used for reagent standardization and control as well as binding standardization and control as well.

When a plastic or glass microfluidic device is used for capture, fluorescence microscopy, bright field microscopy, or a combination thereof can be used to analyze the morphology and/or nuclei of the rare target cells that are labeled with the PE-labeled anti-cytokeratin antibodies and Hoechst stain. Capture efficiency can be measured, e.g., by adding a known number of discrete particles to the sample to be analyzed. For example, the discrete particles can be beads coated with one or more antigens (e.g., Ep-CAM or peptide thereof) and a detectable moiety (e.g., a fluorescent dye), where at least one of the antigens is the same antigen recognized by the binding moiety. In some embodiments, the beads are coated with a second antigen (e.g., a cytokeratin or peptide thereof) that is distinct from the first antigen, and which can be detected (e.g., by immunofluorescence). This allows detection efficiency to be determined separately from capture efficiency. In some embodiments, the beads are coated with a minimum amount of target antigen that is no greater than the capture threshold for the device. In other words, beads coated with the minimum amount of target antigen approximate the “capture characteristics” of target cells (e.g., tumor cells) that express a minimal amount of target antigen.

In other embodiments, discrete particles are detectably labeled cells bearing an antigen recognized by a binding moiety. For example, the cells can be cells from a cancer cell line (e.g., a human advanced lung cancer cell line NCI-H1650; ATCC Number CRL-5883). This cell line has a heterozygous 15 bp in-frame deletion in exon 19 of EGFR that renders it susceptible to gefitinib. Cells from cancer cell lines can be fixed to prolong their shelf life. In some embodiments, a cell line is selected as a source of marker cells based on its average surface expression level of a target antigen (e.g., EpCAM). Cells from confluent cultures can be harvested with trypsin, stained with the vital dye Cell Tracker Orange (CMRA reagent, Molecular Probes, Eugene, Oreg.), re-suspended in fresh whole blood and flowed through the microfluidic chip at various flow rates. After the cells are processed in the capture module, the device is washed through with buffer at a higher flow rate (3 ml/hr) to remove non-specifically bound cells. The spiked-in marker cells or rare target cells (present in the original sample) captured by the device are then detected or enumerated by fluorescence microscopy.

In some embodiments, at least one detectable label (e.g., a fluorophore) that is used to detect discrete particles is distinct from detectable labels used to detect the rare target cells. One of ordinary skill in the art will recognize that many labeling agents (e.g., fluorophores) are known and that combinations of such labeling reagents can be selected to minimize overlap in their detection signals (e.g., emission spectra).

In some embodiments, the fluid sample is spiked with a number of discrete particles having a detectable label for detection in the microfluidic device. In some embodiments, the detectable label is distinct from detectable label detected on the rare cells captured by the microfluidic device. In some embodiments, the detection of the discrete particles indicates whether the microfluidic device is working or not. In some embodiments, the detection of said marked cells or beads indicates the efficiency of the microfluidic device's detection capabilities.

For example, discrete particles spiked into a whole blood sample and recovered by affinity capture as described above can be analyzed in situ to confirm that the device is functioning with a satisfactory capture efficiency on the biological sample being processed. Determining the specific capture efficiency of the device for an individual biological sample permits a more accurate determination of confidence levels for the number of detected cells in the individual sample, as described in more detail below. One advantage of microfluidic devices described herein is that the gentle handling of the cells during processing allows for greater recovery of rare target cells compared to other separation/capture devices.

Evaluating Cell Viability

The methods of the invention provide for evaluating the viability of a circulating tumor cell in an object by analyzing the cell for an ability to perform a function. The function can be the prevention of a transport of an agent into the circulating tumor cell. The agent can be a molecule that can be cell membrane-impermeable.

A method for determining viability of a circulating tumor cell in a sample obtained from a subject can comprise contacting the sample with a cell membrane-impermeable nucleic acid binding agent capable of being photoactivated, exposing the sample to a dose of light to photoactivate the nucleic acid binding reagent, capturing a circulating tumor cell from the sample; and detecting the presence or absence of the nucleic acid binding reagent in the nucleus of the captured circulating tumor cell. The presence of the nucleic acid binding reagent can indicate that the captured tumor cell is not viable. A microfluidic device can be used to capture the circulating tumor cell. The microfluidic device can comprise an array of obstacles and/or one or more binding moieties. The array of obstacles can form a network of gaps between adjacent obstacles and the gaps between adjacent obstacles can be between 1 and 300 microns in length.

Examples of agents that can be used to determine viability of a circulating tumor cell include, but are not limited to, AlamarBlue™, calcein-AM, BCECF AM, Carboxyfluorescein Diacetate, Pentafluorobenzoyl Aminofluorescein Diacetate, Carboxynaphthofluorescein Diacetate, Chloromethyl SNARF-1 Acetate, or C₁₂ resazurin. Examples of inviability reagents include but are not limited to, ethidium bromide, ethidum homodimer-1, propidium iodide, SYTOX Green, SYTOX Orange and SYTOX Blue Nucleic Acid Stains (Invitrogen, Inc., Carlsbad, Calif.), TOTO monomeric cyanine nucleic acid stains, TO-PRO dimeric cyanine nucleic ace stains, photoactivatable fluorescent nucleic acid binding dyes (e.g., ethidium monoazide), or trypan blue.

In some embodiments, a photoactivatable fluorescent nucleic acid binding dye (e.g., ethidium monoazide) is added to a biological sample within about an hour of the time it is obtained from a subject. After addition of the photoactivatable dye, the sample is exposed to a dose of light (e.g., UV light) to photo activate and covalently cross-link nucleic acid-bound dye in cells, thereby permanently marking them. Excess free dye can be washed out shortly before or after the photoactivation step. Thus, rare cells that were dead in the sample shortly after the sample was obtained from the subject can be detected, while avoiding the detection of cells that die during subsequent manipulations of the sample. In some embodiments, a fixative (e.g., formaldehyde or methanol) is added to a biological sample after contacting the sample with a photoactivatable nucleic acid binding dye, and after photo-activation of the dye, but prior to an enrichment step.

Sample Processing

The devices of the invention provide for diagnosing, theranosing, or prognosing a condition in a patient comprising a microfluidic device comprising an array of obstacles and one or more binding moieties that selectively retains one or more rare cells, wherein the microfluidic device is configured for flowing between about 7-1,500, 0.1-1,500, 1-1000, or 1.5-500 mL/hr of blood sample from said patient through said microfluidic device. The one or more binding moieties are anti-EpCAM. The one or more rare cells are circulating tumor cells or epithelial cells. The microfluidic device can contain no more than 50, 100, or 200 μL, of said sample. The microfluidic device comprises no more than one microfluidic device.

Hydrodynamic force can be used to flow the blood sample through the microfluidic device. The hydrodynamic force can be provided for by a pump. The pump can be a peristaltic pump, a syringe pump, or a centrifugal pump. A pressure differential between an inlet of the microfluidic device and an outlet of the microfluidic device may also drive blood flow. The pressure differential can be less than 0.5, 1, 2, 10, 15, 20, 30, 40, 50, 100, 200, 250, or 300 psi. The pressure differential can be greater than 0.5, 1, 2, 10, 15, 20, 30, 40, 50, 100, 200, 250, or 300 psi.

The blood sample flowing through the microfluidic device can experience laminar flow or turbulent flow. The Reynolds number for fluid flowing through the microfluidic device can be less than about 0.01, or between about 0.01 and about 0.0005, or less than about 0.0005.

In yet other embodiments, at least one of the inlet and outlet connects to a flow generator. In some embodiments, the flow generator is connected to the inlet, whereby the flow generator is adapted and configured to drive the fluid sample through the chamber. In other embodiments, the flow generator is connected to the outlet, whereby the flow generator is adapted and configured to pull the fluid sample through the chamber. In one non-limiting embodiment, the flow generator may be a peristaltic pump or a syringe pump. An example of a plurality of devices 200A, 200B, 200C connected to a peristaltic pump 220 is shown in FIG. 2. In other embodiments, the flow generator is adapted and configured to provide at least one of an intermittent liquid sample flow and a continuous liquid sample flow through the chamber.

FIG. 2 depicts is a system 216 of three microfluidic devices 200A, 200B, 200C having arrays 202A, 202B, 202C of obstacles 204A, 204B, 204C, wherein two devices 200A, 200B are configured to flow a single sample 218 in parallel, and wherein the third microfluidic device 200C is adapted and configured to flow the sample in series through the device 200C after the sample has flowed through the first two devices 200A, 200B, whereby the outlets 212A, 212B of the first two devices 200A, 200B, flow to the inlet 210C of the third device 210C, and wherein a peristaltic pump 220 is adapted and configured to flow the sample 218 through the system 216.

FIG. 2 depicts is a system 216 of three microfluidic devices 200A, 200B, 200C having arrays 202A, 202B, 202C of obstacles 204A, 204B, 204C, wherein two devices 200A, 200B are configured to flow a single sample 218 in parallel, and wherein the third microfluidic device 200C is adapted and configured to flow the sample in series through the device 200C after the sample has flowed through the first two devices 200A, 200B, whereby the outlets 212A, 212B of the first two devices 200A, 200B, flow to the inlet 210C of the third device 210C, and wherein a peristaltic pump 220 is adapted and configured to flow the sample 218 through the system 216. The direction of flow is shown by arrows W, X, Y, and the direction that the peristaltic pump, for example, turns is shown by arrow Z. Also shown is a sample reservoir 222, which may include a rocker or another preprocessing system as described herein. Further shown is a container 224 for capturing the sample 218 which has been flowed through the system 216. As discussed herein, there are multiple variations of this system 216 and in a single device 200. For example, other flow generators and placements are contemplated, the flow of the sample or the buffer or both may be continuous or intermittent, the number and the arrangement of devices may be varied, the direction of flow may be varied, the size of the arrays may be varied, the existence and types of binding moieties may be varied, the size, shapes, and arrangements of the obstacles may be varied, the number of times the sample is run through a device or multiple devices may be varied, the amount or existence of a buffer introduced in the system, as well as its flow rate may be varied, the amount and flow rates of the sample may be varied, among other non-limiting variations discussed herein.

The methods of the invention provide for diagnosing, theranosing, or prognosing a condition in a patient comprising: flowing between about 7-1,500, 0.1-1,500, 1-1000, or 1.5-500 mL/hr of blood sample from said patient through a microfluidic device comprising an array of obstacles and one or more binding moieties that selectively retains one or more rare cells; and enriching in one or more rare cells. The one or more binding moieties are anti-EpCAM. The one or more rare cells are circulating tumor cells or epithelial cells. The microfluidic device can contain no more than 50, 100, or 200 μL, of said sample. The microfluidic device comprises no more than one microfluidic device.

Retention of Rare Cells

The devices of the invention provide for a device configured for enriching one or more rare cells from a sample obtained from a patient comprising a microfluidic device including a capture array of obstacles covered with binding moieties to selectively retain said rare cells and a separation array of obstacles covered with binding moieties to selectively retain said rare cells. At least 1, 5, 10, 25, 50 or 75% of said rare cells can be retained within at least the first 30 rows of said capture array of obstacles. The sample can be at least 50, 75, or 100 times greater than an interior volume of the microfluidic device.

The microfluidic device with a capture array and a separation array can be used to enrich circulating tumor cells. The capture array of obstacles can be fluidly coupled to the separation array of obstacles and is positioned such that said sample contacts said separation array of obstacles prior to contacting said capture array of obstacles. The capture array of obstacles can comprise a network of gaps with an average capture gap length between adjacent obstacles and the separation array of obstacles can comprise a network of gaps with an average separation gap length between obstacles. The average capture gap length can be no more than 20 microns and the average separation gap length can be no less than 20 microns. The average capture gap length can be less than the average separation gap length. The binding moieties can comprise anti-EpCAM, anti-EGFR, anti-LAR, or anti-cytokeratin.

The methods of the invention provide for enriching one or more rare cells from a sample obtained from a patient comprising flowing said sample through a microfluidic device including a capture array of obstacles covered with binding moieties to selectively retain said rare cells and a separation array of obstacles covered with binding moieties to selectively retain said rare cells. At least 1, 5, 10, 25, 50 or 75% of said rare cells can be retained within at least the first 30 rows of said capture array of obstacles. The sample can be at least 50, 75, or 100 times greater than an interior volume of the microfluidic device. The rare cells can be circulating tumor cells.

The method for enriching one or more rare cells using a microfluidic device including a capture array and a separation array can further comprise analyzing the enriched rare cells. The analysis methods can include enumerating, labeling, or imaging said rare cells. The results of the analysis methods can be used diagnose, theranose, or prognose a condition in the patient.

Methods for Diagnosing, Prognosing, or Theranosing

The methods of the invention can comprise diagnosing, prognosing, or theranosing based on the analysis methods described herein. The methods for diagnosing, prognosing, or theranosing can comprise obtaining a sample from a patient, analyzing a sample obtained from a patient, enriching a sample obtained from a patient for one or more cells, and/or analyzing one or more cells enriched from a sample obtained from a patient.

Diagnosing can comprise determining the condition of a patient. For example, a patient can be diagnosed with cancer or with another disease based on results from obtaining a sample from the patient, enriching a sample in one or more rare cells, and analyzing the one or more rare cells.

Prognosing can comprise determining the outcome of a patient's disease, the chance of recovery, or how the disease will progress. For example, a patient can obtain a prognosis of having a 50% chance of recovery based on results from obtaining a sample from the patient, enriching a sample in one or more rare cells, and analyzing the one or more rare cells.

Theranosis can comprise determining a therapy treatment. For example, a patient's therapy treatment can be chosen based on the response of one or more enriched cells that have been cultured and treated with a therapeutic agent.

Patients and Computer Systems

The invention contemplates treatment human and non-human patients. The patient can be a human or an animal.

Any of the steps herein can be performed using computer program product that comprises a computer executable logic recorded on a computer readable medium. For example, the computer program can process data from the analysis of target genomic DNA regions to determine the presence or absence of cancer cells in a sample and to determine one or more abnormalities in cells detected. For example, the number of cells or properties of cells can be determined using a computer program and algorithms. In some cases, computer executable logic uses data input on STR or SNP intensities to determine the presence of cancer cells in a test sample and determine abnormalities and/or conditions in said cells.

Specific Obstacle Arrangements

The enrichment devices herein can have various obstacle arrangements, sizes, and shapes.

Any of the devices herein can have at least one obstacle with a cross-sectional shape that is a circle, an oval, a diamond, a triangle, a kidney, an arc, or a ‘c’. A ‘c’ shape appears like the letter ‘c’. The device may have different shaped obstacles or can have obstacles of uniform shape.

An array can have a subset of obstacles or an average obstacle with a diameter of at least 20 μm, at most 400 μm, a range of about 20 μm to about 400 μm, a range of about 40 μm to about 160 μm, and a range of about 60 μm to about 120 μm. When referring to column obstacle diameter, “about” refers to variations in diameter of 1 μm to 5 μm or of 5 μm to 10 μm.

In some instances, an enrichment device has a subset of obstacles with a height of or an average obstacle height of at least about 10 μm, at most about 200 μm, between about 50 μm and about 150 μm, between about 75 μm and about 125 μm. When referring to obstacle height, “about” refers to variations in height of 1 μm to 2 μm or of 2 μm to 5 μm.

Provided herein is a microfluidic device for enriching one or more rare cells from a fluid sample comprising rare cells and non-rare cells, the device comprising a chamber having a base layer, a first array of obstacles arising from the base layer, and a plurality of gaps between obstacles. The first array can have a size at most about 2.0 cm in width and about at most about 6.0 cm long, at most about 1 cm in width and at most about 3 cm long, at most about 1 cm in width and at most about 1.5 cm long, at most about 6 cm in width and at most about 10 cm long. Typically, an array is no larger than the size of a standard microscope slide.

The microfluidic device can comprise a fluid channel. The fluid channel can allow for flow of sample microfluidic device. In some embodiments the fluid channel has a height of, for example, at least about 10 μm, at most about 200 μm, between about 50 μm and about 150 μm, between about 75 μm and about 125 μm. When referring to fluid channel height, “about” refers to variations in height of 1 μm to 5 μm or of 5 μm to 10 μm.

In some embodiments, the device comprises at least one of a second array, third array, and fourth array of obstacles arising from the base layer. These additional arrays may be positioned in series or in parallel. There may be dividers between the arrays, or the arrays may be in fluid communication.

In other embodiments the at least one of a second array, third array, and fourth array has a size of, for example, at most about 2.0 cm in width and about at most about 6.0 cm long, at most about 1 cm in width and at most about 3 cm long, at most about 1 cm in width and at most about 1.5 cm long, at most about 6 cm in width and at most about 10 cm long. In other embodiments, the first array is adjacent to the second array and wherein the chamber comprises a divider for separating the fluid sample in the first array from the fluid sample in the second array. In some embodiments having a first array, a second array, and optional additional arrays, each of said obstacles has a surface providing binding moiety, said binding moiety attached to said surface of said structure via a cleavable linker and capable of specifically binding said rare cells.

In some instances, an enrichment device comprises an array of obstacles in chamber having a volume free of obstacles selected from the group of at most about 1.2 cubic centimeters, about 0.054 cubic centimeters, and at least about 0.0015 cubic centimeters. When referring to chamber volume, “about” refers to variations in chamber volume of 0.0005 cubic centimeters to 0.001 cubic centimeters or of 0.005 cubic centimeters to 0.01 μm.

In some embodiments, the microfluidic device can hold a volume of fluid including, for example, at least 10 μL, at most 500 μL, between about 10 μL, and about 500 μL, between about 20 μL, and about 300 μL, between about 30 μL and about 100 μL, and between about 40 μL and about 60 μL. When referring to the volume of fluid the chamber can hold, “about” refers to variations in volume of 5 μL, to 50 μL or of 1 mL to 10 mL.

Provided herein is a device for selectively enriching rare cells comprising a chamber comprising an array of obstacles functionalized to selectively bind epithelial cells, wherein said chamber can hold at least at least 10 μL, of a fluid.

Depicted in FIG. 1A is a microfluidic device 100 having an array 102 of obstacles 104, a lid 106 and removable threaded screw ports 108A, 108B attached to the inlet 110 and to the outlet 112. Some portion of the array 102, the base layer 114, or the lid 106 may be coated with one or more binding moieties for capture of one or more rare cells. Alternatively, or in addition, the geometry and features of the device 100 and the flow of sample 118 and buffer through the device 100 may result in capture of one or more rare cells based on size. Depicted in FIG. 1B is a cross-sectional view of the microfluidic device 100 of FIG. 1A having a lid 106 and removable screw ports 108A, 108B, cut along line B-B of FIG. 1A. The device 100 and the array 102 may be transparent, and the lid 106 may also be transparent for rare cell analysis or enumeration directly on the device 100. Removing the screw ports 108A, 108B allow for enumeration, processing, or analysis of the captured one or more rare cells directly on the device 100 using methods and processes provided herein. The device 100 may be made from various materials, including, but not limited to, glass, plastic or silicon.

In some instances, an enrichment device comprises an array of obstacles wherein the device comprises an inlet port at one end of the device, and an outlet port at the opposite end of the device, and wherein the gaps decrease in size from the inlet port to the outlet port. Such decrease may be continuous or the device may have several stages, each stage with a particular and smaller gap size.

The microfluidic device with an array of obstacles can comprise an inlet port for fluid flow into the microfluidic device, an outlet port for fluid flow out of the microfluidic device, uniformly arranged obstacles, or non-uniformly arranged obstacles. The microfluidic device can comprise one or more rows of obstacles, where a row of obstacles is staggered relative to an adjacent row of obstacles.

A device can comprise: a first array of obstacles having a restricted gap dispersed in a uniform pattern therein coupled to a second array of obstacles having a uniform pattern of obstacles and no restricted gap. In some embodiments, the restricted gap has a distance between adjacent obstacles of between about 10 μm and about 20 μm, and a second gap size having a distance between adjacent obstacles selected from the group of a minimum of about 5 μm, between about 5 μm and about 80 μm, between about 40 μm and about 60 μm, and a maximum of about 100 μm.

In some instances, an enrichment device comprises an array of obstacles with a first gap between at least two obstacles, wherein the first gap is, for example, a minimum of about 5 μm, between about 5 pin and about 80 μm, between about 10 μm and about 20 μm, between about 20 μm and about 40 μm, between about 40 μm and about 60 μm, between about 60 and about 80 μm, or a maximum of about 100 μm. When referring to gap size, “about” refers to variations in gap size of up to 1 μm or up to 2 μm.

Such a device can optionally comprise a second gap between at least two obstacles, wherein the second gap is, for example, a minimum of about 5 μm, between about 5 μm and about 80 μm, between about 10 μm and about 20 μm, between about 20 μm and about 40 μm, between about 40 μm and about 60 μm, between about 60 and about 80 μm, or a maximum of about 100 μm. When referring to gap size, “about” refers to variations in gap size of up to 1 μm or up to 2 μm.

In some embodiments, at least 10%, 20%, 30%, 40%, or 50% 60%, 70%, 80%, or 90% of all gaps between adjacent obstacles consist of a first gap size. In some embodiments, at least 10%, 20%, 30%, 40%, or 50% 60%, 70%, 80%, or 90% of all gaps between adjacent obstacles consist of a second gap size. The second gap size may be distributed throughout the device in a pattern or randomly. The first gap can be narrower than the second gap or the second gap can be narrower than the first gap. The narrower gap can help to capture target cells.

A microfluidic device for enriching one or more rare cells from a fluid sample comprising rare cells and non-rare cells, the device comprising a chamber having a base layer, an array of obstacles arising from the base layer, a plurality of gaps between obstacles, wherein the device comprises a relatively similar proportion of narrow gaps and wide gaps to total gaps or total number of obstacles. The narrow gaps can be distinguished from the wide gaps by having gaps of smaller length between two obstacles. In some embodiments, a narrow gap has a gap length smaller than an average gap and a wide gap has a gap length larger than the average gap.

A device can comprise a first gap having a distance between adjacent obstacles selected from the group of a minimum of about 5 μm, between about 5 μm and about 80 μm, between about 10 μm and about 20 μm, and a maximum of about 100 μm, and a second gap size having a distance between adjacent obstacles selected from the group of a minimum of about 5 μm, between about 5 μm and about 80 μm, between about 40 μm and about 60 μm, and a maximum of about 100 μm.

A microfluidic device for enriching one or more rare cells from a fluid sample can comprise a chamber having a base layer, an array of obstacles arising from the base layer, a first gap between at least two obstacles, wherein the first gap is at least one of a minimum of about 5 μm, and a maximum of about 100 μm, wherein the array of obstacles comprises between about 200 and about 2,000,000 obstacles, and wherein the chamber has a volume free of obstacles of at least about 0.0015 cubic centimeters. In some embodiments the chamber has a volume free of obstacles of at most 0.10 cubic centimeters.

In one non-limiting embodiment of the device at least two obstacles are arranged in a repeating pattern. In another non-limiting embodiment of the device at least two obstacles are arranged in an annular pattern. In yet another non-limiting embodiment of the device wherein at least one obstacle has a defined cross-sectional shape as described herein, and optionally arranged in a pattern described herein, each of said obstacles has a surface allowing for a binding moiety.

An enrichment device can have between about 1000 and 15000 first gaps, and between about 5000 and about 10000 first gaps is provided. A device comprising a first gap having a distance between adjacent obstacles selected from the group of a minimum of about 5 μm, between about 5 μm and about 80 μm, between about 10 μm and about 20 μm, and a maximum of about 100 μm is also provided. Such devices, or any device described herein may be used with the methods described herein.

Any of the devices herein can have a chamber for the obstacles that can hold between about 200 and about 2,000,000 obstacles, between about 200 and about 5,000 obstacles, between about 5,000 and about 10,000 obstacles, between about 10,000 and about 50,000 obstacles, between about 50,000 and about 100,000 obstacles, between about 100,000 and about 150,000 obstacles, between about 150,000 and about 300,000 obstacles, between about 300,000 and about 500,000 obstacles, between about 500,000 and about 2,000,000 obstacles. In referring to the number of obstacles, “about” refers to variations in number of obstacles of 1 to 50 obstacles, or of 100 to 500 obstacles. Where multiple arrays are used, each array may have a separate chamber, on separate devices or on a single device, wherein each array may have the number and density of obstacles disclosed herein.

The array of obstacles in some embodiments comprises between about 1 obstacle per square millimeter and about 400 obstacles per square millimeter, between about 10 and about 350 obstacles per square millimeter, between about 25 and about 300 obstacles per square millimeter, between about 35 and about 250 obstacles per square millimeter, between about 45 and about 200 obstacles per square millimeter, between about 55 and about 150 obstacles per square millimeter, between about 65 and about 100 obstacles per square millimeter, and between about 75 and about 95 obstacles per square millimeter. In referring to the number of obstacles per obstacle area, “about” refers to variations in number of obstacles per obstacle area of 1 to 5 obstacles per square millimeter, or of 10 to 20 obstacles square millimeter.

Some embodiments provide a device for selectively enriching rare cells comprising a chamber having an array of obstacles that selectively binds epithelial cells over non-epithelial cells, wherein said array of obstacles has a surface area of at least 100 mm̂2, between about 1000 mm̂2 and about 10000 mm̂2, between about 1000 mm̂2 and about 1500 mm̂2, between about 1500 mm̂2 and about 2000 mm̂2, between about 2000 mm̂2 and about 2500 mm̂2, between about 2500 mm̂2 and about 3000 mm̂2, between about 3000 mm̂2 and about 3500 mm̂2, between about 3500 mm̂2 and about 5000 mm̂2, between about 5000 mm̂2 and about 10000 mm̂2, between about 10000 mm̂2 and about 15000 mm̂2, between about 15000 mm̂2 and about 35000 mm̂2, and at most 35000 mm̂2, wherein the surface area of the obstacles that selectively binds epithelial cells includes the top of the obstacles. Some embodiments provide a device for selectively enriching rare cells comprising a chamber having an array of obstacles that selectively binds epithelial cells over non-epithelial cells, wherein said array of obstacles has a surface area of at least 100 mm̂2, between about 1000 mm̂2 and about 10000 mm̂2, between about 1000 mm̂2 and about 1500 mm̂2, between about 1500 mm̂2 and about 2000 mm̂2, between about 2000 mm̂2 and about 2500 mm̂2, between about 2500 mm̂2 and about 3000 mm̂2, between about 3000 mm̂2 and about 3500 mm̂2, between about 3500 mm̂2 and about 5000 mm̂2, between about 5000 mm̂2 and about 10000 mm̂2, between about 10000 mm̂2 and about 15000 mm̂2, between about 15000 mm̂2 and about 35000 mm̂2, and at most 35000 mm̂2, wherein the surface area of the obstacles that selectively binds epithelial cells does not include the top of the obstacles.

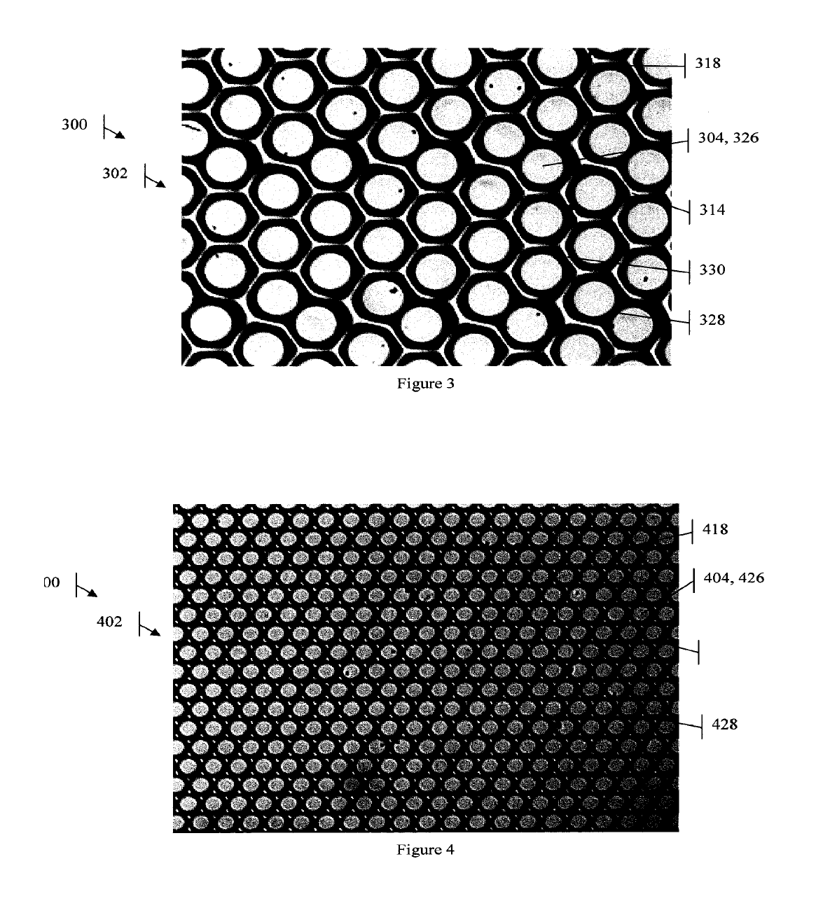

FIG. 3 depicts a zoomed-in view of a sample 318 flowing through an array 302 of obstacles 304 in a microfluidic device 300 having generally columnar obstacles 326 having a height of at least about 10 μm, at most about 200 μm, between about 50 μm and about 150 μm, between about 75 μm and about 125 μm about 100 μm, and a diameter of at least about 10 μm, at most about 200 μm, between about 75 μm and about 150 μm, and between 75 μm and about 200 μm, wherein the array 302 of obstacles 304 is at most about 2.0 cm in width and at most about 6.0 cm long, wherein the sample 318 flow rate is between about 0.5 mL/hr and about 2.0 mL/hr or between about 5 μL/min and about 50 μL/min, wherein the buffer wash flow rate is between about 1 mL/hr and about 20 mL/hr or between about 10 μL/min and 200 μL/min, wherein the total number of obstacles 304 is about between about 200 and about 2,000,000 obstacles, between about or between about 50,000 and about 100,000 obstacles, wherein device 302 has a first gap 328 size between adjacent obstacles 304 of a minimum of about 5 μm, between about 5 μm and about 80 μm, between about 10 μm and about 20 μm, or a maximum of about 100 μm, and a second gap 330 size between adjacent obstacles of a minimum of about 5 μm, between about 5 μm and about 80 μm, between about 40 μm and about 60 μm, or a maximum of about 100 μm, wherein there are between about 1000 and 15000, or between about 5000 and about 10000 first gaps 328, and wherein along a single path from the inlet (not shown) to the outlet (not shown) of the device 300, at least between about 100 and about 2000, or between about 200 and about 1000 obstacles are encountered. In this example, the array 302 volume is, for example, at least 10 μL, at most 500 μL, between about 10 μL and about 500 μL, between about 20 μL and about 300 μL, between about 30 μL and about 100 μL, and between about 40 μL and about 60 μL, the surface area of the portion of the surface having binding moieties (i.e. the circumference of each obstacle 304 and on the portions of the base layer 314 that are free of obstacles 304) is at least 100 mm̂2, between about 1000 mm̂2 and about 10000 mm̂2, between about 2000 mm̂2 and about 2500 mm̂2, and at most 35000 mm̂2.

FIG. 4 depicts a zoomed-in view of a sample 418 flowing through an array 402 of obstacles 404 in a microfluidic device 400 having generally columnar obstacles 426 having a height of any other embodiment described herein, a diameter of at least about 10 μm, at most about 200 μm, between about 20 μm and about 50 μm, wherein the array 402 of obstacles 404 can have a width and length of any other embodiment described herein, wherein the flow rate of the sample 418 and the buffer can be that of any other embodiment described herein, wherein the total number of obstacles 404 is between about 200 and about 2,000,000 obstacles, or between about 300,000 and about 500,000 obstacles, wherein device 400 has a gap size 428 between adjacent obstacles 404 of a minimum of about 5 μm, between about 5 μm and about 80 μm, or between about 10 μm and about 20 μm. In this example, the array 402 volume can be that of any other embodiment described herein, the surface area of the portion of the surface having binding moieties (i.e. the circumference of each obstacle 404 and on the portions of the base layer 414 that are free of obstacles 404) at least 100 mm̂2, between about 1000 mm̂2 and about 10000 mm̂2, between about 3500 mm̂2 and about 5000 mm̂2, and at most 35000 mm̂2,

FIG. 5 depicts a zoomed-in view of a sample 518 flowing through an array 502 of obstacles in a microfluidic device 500 having generally columnar obstacles 426 having a height of of any other embodiment described herein, and a diameter of at least about 10 μm, at most about 200 μm, between about 50 μm and about 100 μm, wherein the array 502 of obstacles 504 can have a width and length of any other embodiment described herein, wherein the flow rate of the sample 518 and the buffer can be that of any other embodiment described herein, wherein the total number of obstacles 504 is between about 200 and about 2,000,000 obstacles, between about 100,000 and about 150,000 obstacles, wherein device 500 has a gap size 528 between adjacent obstacles 504 of a minimum of about 5 μm, between about 5 μm and about 80 μm, between about 20 μm and about 40 μm, or a maximum of about 100 μm, about 31 μm. In this example, the array 502 volume can be that of any other embodiment described herein, the surface area of the portion of the surface having binding moieties (i.e. the circumference of each obstacle 504 and on the portions of the base layer 514 that are free of obstacles 504) is at least 100 mm̂2, between about 1000 mm̂2 and about 10000 mm̂2, between about 3000 mm̂2 and about 3500 mm̂2, and at most 35000 mm̂2.

FIG. 6 depicts a zoomed-in view of a sample 618 flowing through an array 602 of obstacles 614 in a microfluidic device 600 having generally columnar obstacles 626 having a height of any other embodiment described herein, a diameter of at least about 10 μm, at most about 200 μm, between about 20 μm and about 50 μm, wherein the array and a diameter of any other embodiment described herein, wherein the array 602 can have a width and length of any other embodiment described herein, wherein the flow rate of the sample 618 and the buffer can be that of any other embodiment described herein, wherein the total number of obstacles 604 is between about 200 and about 2,000,000 obstacles, or between about 50,000 and about 100,000 obstacles, wherein device 600 has a gap size 628 between obstacles 604 of a minimum of about 5 μm, between about 5 μm and about 80 μm, between about 40 μm and about 60 μm, or a maximum of about 100 μm. In this example, the array 602 volume can be that of any other embodiment described herein, the surface area of the portion of the surface having binding moieties (i.e. the circumference of each obstacle 604 and on the portions of the base layer 614 that are free of obstacles 604) is at least 100 mm̂2, between about 1000 mm̂2 and about 10000 mm̂2, between about 2000 mm̂2 and about 2500 mm̂2, and at most 35000 mm̂2

FIG. 7 depicts a zoomed-in view of a sample 718 flowing through an array 702 of obstacles 704 in a microfluidic device 700 having generally half-circular obstacles 732 having a height of any other embodiment described herein and a length across the long straight edge of the half-circle of at least about 10 μm, at most about 200 μm, or between 75 μm and about 200 μm, wherein the array 702 of obstacles 704 can have a width and length of any other embodiment described herein, wherein the flow rate of the sample 718 and the buffer can be that of any other embodiment described herein, wherein the total number of obstacles 704 is between about 200 and about 2,000,000 obstacles, or between about 10,000 and about 50,000 obstacles, wherein device 700 has a gap size 728 between obstacles 704 of a minimum of about 5 μm, between about 5 μm and about 80 μm, between about 20 μm and about 40 μm, between about 40 μm and about 60 μm, or a maximum of about 100 μm. In this example the array 702 volume is at least 10 μL, at most 500 μL, between about 10 μL and about 500 μL, between about 20 μL and about 300 μL, or between about 30 μL and about 100 μL, the surface area of the portion of the surface having binding moieties (i.e. the circumference of each obstacle 704 and on the portions of the base layer 714 that are free of obstacles 704) is at least 100 mm̂2, between about 1000 mm̂2 and about 10000 mm̂2, between about 2000 mm̂2 and about 2500 mm̂2, and at most 35000 mm̂2.

FIG. 8 depicts a zoomed-in view of a sample 818 flowing through an array 802 of obstacles 804 in a microfluidic device 800 having generally columnar obstacles 826 having a height of any other embodiment described herein and a diameter of any other embodiment described herein, wherein the array 802 of obstacles 804 can have a width and length of any other embodiment described herein, wherein the flow rate of the sample 818 and the buffer can be that of any other embodiment described herein, wherein the total number of obstacles 804 is between about 200 and about 2,000,000 obstacles, or between about 50,000 and about 100,000 obstacles, wherein device 800 has a first gap 828 size between adjacent obstacles of a minimum of about 5 μm, between about 5 μm and about 80 μm, between about 10 μm and about 20 μm, or a maximum of about 100 μm, and a second gap 830 size between adjacent obstacles a minimum of about 5 μm, between about 5 μm and about 80 μm, between about 20 μm and about 40 μm, or a maximum of about 100 μm. In this example, the array 802 volume is at least 10 μL, at most 500 μL, between about 10 μL, and about 500 μL, between about 20 μL and about 300 μL, between about 30 μL and about 100 μL, the surface area of the portion of the surface having binding moieties (i.e. the circumference of each obstacle 804 and on the portions of the base layer 814 that are free of obstacles 804) is at least 100 mm̂2, between about 1000 mm̂2 and about 10000 mm̂2, between about 2500 mm̂2 and about 3000 mm̂2, and at most 35000 mm̂2.

FIG. 9 depicts a zoomed-in view of a sample 918 flowing through an array 902 of obstacles 904 in a microfluidic device 900 having generally columnar obstacles 926 having a height of any other embodiment described herein and a diameter of any other embodiment described herein, wherein the array 902 of obstacles 904 can have a width and length of any other embodiment described herein, wherein the flow rate of the sample 918 and the buffer can be that of any other embodiment described herein, wherein the total number of obstacles 904 is between about 200 and about 2,000,000 obstacles, or between about 50,000 and about 100,000 obstacles, wherein device 900 has a first gap 928 size between adjacent obstacles 904 of a minimum of about 5 μm, between about 5 μm and about 80 μm, between about 20 μm and about 40 μm, or a maximum of about 100 μm, and a second gap 930 size between adjacent obstacles 904 of a minimum of about 5 μm, between about 5 μm and about 80 μm, between about 40 μm and about 60 μm, or a maximum of about 100 μm, and wherein there are between about 1,000 and 25,000, or between about 5,000 and about 10,000 first gaps 928. In this example, the array 902 volume can be that of any other embodiment described herein, the surface area of the portion of the surface having binding moieties (i.e. the circumference of each obstacle 904 and on the portions of the base layer 914 that are free of obstacles 904) is at least 100 mm̂2, between about 1000 mm̂2 and about 10000 mm̂2, between about 2000 mm̂2 and about 2500 mm̂2, and at most 35000 mm̂2.

FIG. 10 depicts a zoomed-in view of a sample 1018 flowing through an array 1002 of obstacles 1004 in a microfluidic device 1000 having generally columnar obstacles 1026 having a height of any other embodiment described herein and a diameter of any other embodiment described herein, wherein the array 1002 of obstacles 1004 can have a width and length of any other embodiment described herein, wherein the flow rate of the sample 1018 and the buffer can be that of any other embodiment described herein, wherein the total number of obstacles 1004 is between about 200 and about 2,000,000 obstacles, between about 50,000 and about 100,000 obstacles, wherein device 1000 has a first gap 1028 size between adjacent obstacles 1004 of a minimum of about 5 μm, between about 5 μm and about 80 μm, between about 10 μm and about 20 μm, or a maximum of about 100 μm, and a second gap 1030 size between adjacent obstacles 1004 of a minimum of about 5 μm, between about 5 μm and about 80 μm, between about 40 μm and about 60 μm, or a maximum of about 100 μm, and wherein there are between about 1,000 and 25,000, or between about 5,000 and about 20,000 first gaps 1028. In this example, the array 1002 volume can be that of any other embodiment described herein, the surface area of the portion of the surface having binding moieties (i.e. the circumference of each obstacle 1004 and on the portions of the base layer 1014 that are free of obstacles 1004) is at least 100 mm̂2, between about 1000 mm̂2 and about 10000 mm̂2, between about 2000 mm̂2 and about 2500 mm̂2, and at most 35000 mm̂2.

Systems

In some embodiments of the system described herein, the system further comprises a sample preparation system wherein said sample preparation system comprises at least one of a rocker, a centrifuge, a negative selection filter, a cell lysis process.

Labeling

Examples of labeling reagents that can be used to label cells of interest include, but are not limited to, antibodies, quantum dots, phage, aptamers, fluorophore-containing molecules, nucleic acid binding agents, enzymes capable of carrying out a detectable chemical reaction, or functionalized beads. Generally, a labeling reagent is smaller than a cell of interest, or a cell of interest bound to a bead; thus, when a cellular sample combined with a labeling reagent is introduced to the device, free labeling reagent moves through the device undeflected and emerges from one or more outlet ports, while bound labeling reagent is retained with the cells. Labeling of a sample prior to introduction to the device can facilitate downstream sample analysis without the need for a release step or destructive methods of analysis. Non-target cells do not interfere with downstream sample analysis that relies on detection of the bound labeling reagent, because this reagent binds selectively to cells of interest.

In some embodiments of the invention, the enrichment of one or more cells is enhanced. For example, one or more cells can be labeled with immunoaffinity beads, thereby increasing the size of the one or more cells. In the case of epithelial cells, e.g., circulating tumor cells, this can further increase their size and thus result in more efficient enrichment. Alternatively, the size of smaller cells can be increased to the extent that they become the largest objects in solution or occupy a unique size range in comparison to the other components of the cellular sample, or so that they co-purify with other cells. The hydrodynamic size of a labeled target cell can be at least 10%, 100%, or even 1,000% greater than the hydrodynamic size of such a cell in the absence of label. Beads can be made of polystyrene, magnetic material, or any other material that can be adhered to cells. Such beads can be neutrally buoyant so as not to disrupt the flow of labeled cells through the device of the invention.

The analysis methods can include nucleic acid analysis, protein analysis, or lipid analysis. The analysis methods can also include analysis of one or more of cell enumeration, cell morphology, pleomorphism, somatic mutation, cell adhesion, cell migration, binding, division, protein phosphorylation, protein glycosylation, mitochondrial abnormalities, cell profiling, genetic profiling, or telomerase activity or levels of a nuclear matrix protein.

In some embodiments, cell enumeration results in an accurate determination of the number of target cells in the sample being analyzed. In order to produce accurate quantitative results, a surface antigen being targeted on the cells of interest typically has known or predictable expression levels, and the binding of the labeling reagent should proceed in a predictable manner, free from interfering substances. Thus, methods of the invention that result in highly enriched cellular samples prior to introduction of labeling reagent are useful. In addition, labeling reagents that allow for amplification of the signal produced can be used because of the low incidence of target cells, such as epithelial cells (e.g., CTCs), in the bloodstream. Reagents that allow for signal amplification include enzymes and phage. Other labeling reagents that do not allow for convenient amplification but nevertheless produce a strong signal, such as quantum dots, can also be used in the methods of the invention.

The ratio of two cells types in the sample, e.g., the ratio of cancer cells to endothelial cells, can be determined. This ratio can be a ratio of the number of each type of cell, or alternatively it can be a ratio of any measured characteristic of each type of cell.

Analysis techniques to perform the methods of analysis can include a variety of analytical techniques. In some embodiments of the invention, a label can be used to detect a component of a cellular sample. The label can be a label conjugated to an antibody that targets any marker listed in Table 1. The label can bind to an analyte, be internalized, or be absorbed. Labels can include detectable labels. The detectable label can be detected based on electromagnetics, mechanical properties, electrical properties, shape, morphology, color, fluorescence, luminescence, phosphorescence, absorbance, magnetic properties, or radioactive emission or any combination thereof.

Light sensitive labels can include quantum dots, fluorescent dyes, or light absorbing molecules. Fluorescent dyes can include Cy dyes, Alexa dyes, or other fluorophore-containing molecules. Quantum dots, e.g., Qdots® from QuantumDot Corp., can also be utilized as a label. Qdots are resistant to photobleaching and can be used in conjunction with two-photon excitation measurements. Fluorescent dyes can then be detected using a fluorometer or a fluorescent microscope.

A label can possess covalently bound enzymes that cleave a substrate. The substrate, once cleaved, can have an altered absorbance at a given wavelength. The extent of cleavage can then be quantified with a spectrometer. Colorimetric or luminescent readouts are possible, depending on the substrate used. In some embodiments of the invention, a measured signal can be above a threshold of detectability. The use of an enzyme label can allow for significant amplification of the measured signal and can lower the threshold of detectability.

Thus the present invention relates to kits comprising one or more of the enrichment modules herein as well as a set of labels selected from any of the labels described above.

### Example 1

### Subarrays

A blood sample obtained from a healthy subject was spiked with cultured H1650 cells (a lunger cancer line). The blood sample was applied to a microfluidic device comprising an array of obstacles and binding moieties to EpCam. The array of obstacles comprises more than one row of obstacles, wherein adjacent rows of obstacles are staggered from each other, as shown in FIG. 3, FIG. 4, FIG. 5, FIG. 6, FIG. 7, FIG. 8, FIG. 9, and FIG. 10.

The microfluidic device had seven subarrays arranged such that the blood sample sequentially contacted the first, second, third, fourth, fifth, sixth, and seventh subarray in order. The seven subarrays had gap lengths between adjacent obstacles of 40, 33, 27, 22, 18, 15, and 12 microns.

The blood sample flow rate through the microfluidic device was either 3.0 mL/hr or 1.5 mL/hr. Rare cells were captured on the microfluidic device and stained with anti-cytokeratin-phycoerythrin and Hoescht dye, scanned using an imaging device to produce a capture plot, and then the capture plot was evaluated to determine if a labeled particle was a rare cell or not. The labeled particles can be rejected, putative, or certitude. Rejected particles are not rare cells, putative particles are of uncertain status, and certitude particles are rare cells.

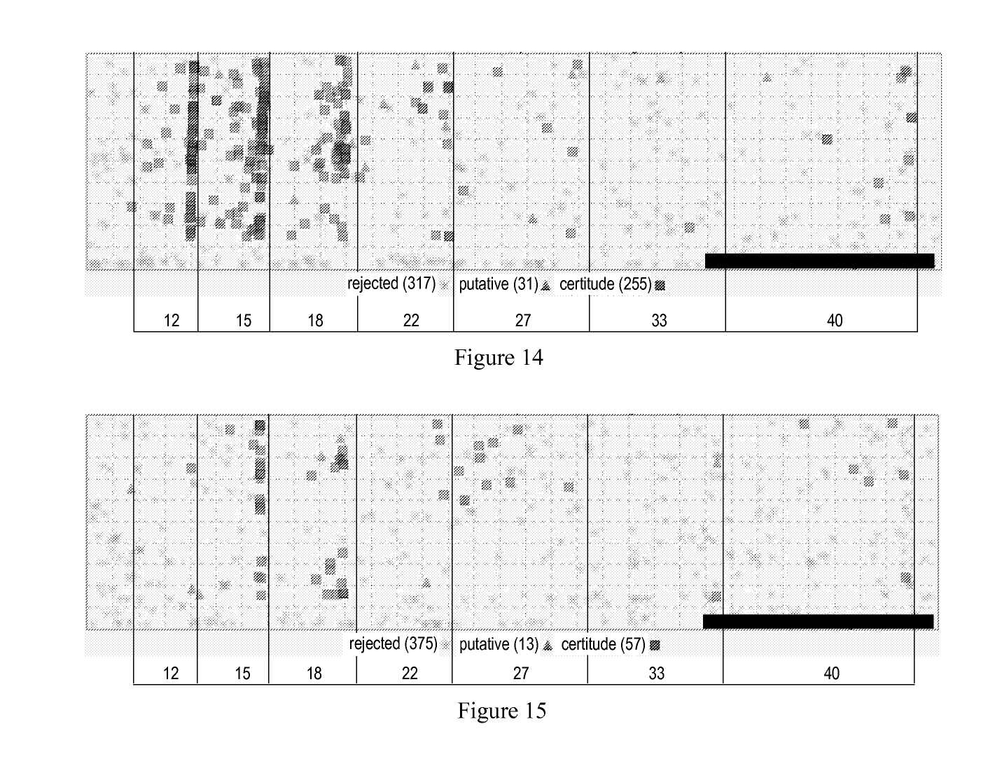

FIG. 12, FIG. 13, and FIG. 14 show capture plots for blood samples that were contacted with the microfluidic device and passed through the microfluidic device at a rate of 3.0 mL/hr. The capture plots show that a high percentage of rare cells were retained in the fifth, sixth, and seventh subarrays. Moreover, an even higher percentage of rare cells were retained in the first few rows of obstacles of the fifth, sixth, and seventh subarrays.

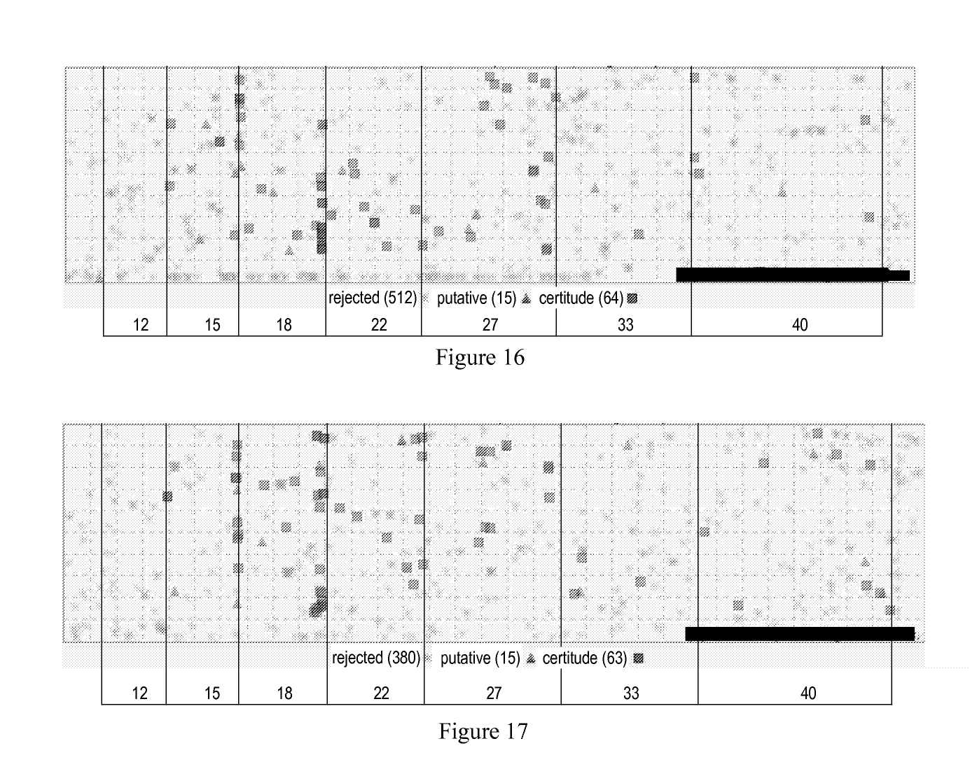

FIG. 15, FIG. 16, and FIG. 17 show capture plots for blood samples that were contacted with the microfluidic device and passed through the microfluidic device at a rate of 1.5 mL/hr. A higher percentage of cells were recovered in earlier subarrays as compared to when the sample was passed through the device at a rate of 3.0 mL/hr.

### Example 2

### Incubating Sample

Three blood samples containing one or more rare cells were applied and incubated with a microfluidic device comprising an array of obstacles and binding moieties to EpCAM for 0, 15, and 30 minutes.

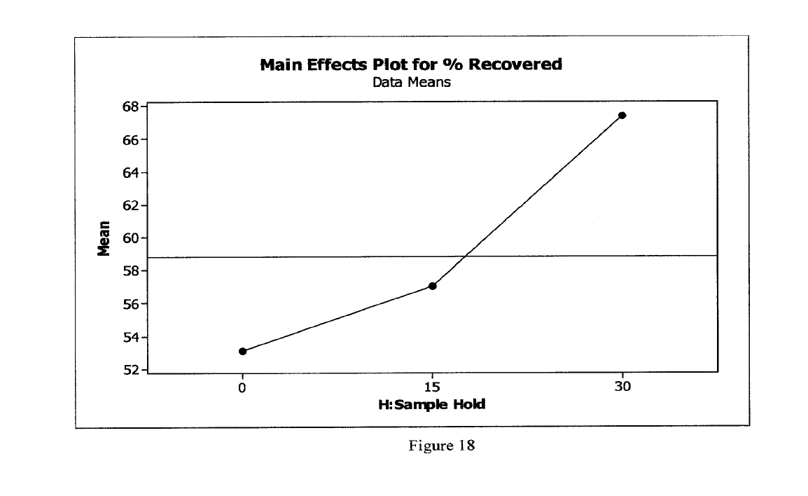

The recovery of rare cells is shown in FIG. 18, where the x-axis shows the sample hold time or incubation time in minutes and the y-axis shows the percent of rare cells recovered as a percentage of a maximum cells recovered by the microfluidic device.

An increase in cell recovery was seen for that maximum incubation time evaluated, which was 30 minutes.

### Example 3

### Internal Standard to Evaluate Reagents and Microfluidic Devices

Discrete particles functionalized with a) EpCAM or b) cytokeratin are passed through a microfluidic device comprising an array of obstacles that are functionalized with binding moieties to EpCAM. The array of obstacles forms a network of gaps for retaining and separating particles in a size range of about 4 to about 100 microns. The discrete particles functionalized with EpCAM are fluorescently labeled with a first dye. The discrete particles functionalized with cytokeratin are not fluorescently labeled. Instead, the discrete particles functionalized with cytokeratin are detected using a fluorescently labeled antibody to cytokeratin. The fluorescently labeled discrete particles and the fluorescently labeled antibody to cytokeratin have fluorescence emission wavelengths that are separated by at least 40 nm.

The discrete particles contact the array of obstacles as they pass through the microfluidic device. Antibodies to EpCAM retain the discrete particles functionalized with EpCAM. The discrete particles that are functionalized with cytokeratin are larger than the gaps in the array of obstacles and become are retained by the array of obstacles. The fluorescently labeled antibody to cytokeratin is passed through the microfluidic device such that they can bind to the discrete particles functionalized with cytokeratin. Excess fluorescent label is washed away by introducing a wash buffer to the microfluidic device.

The microfluidic device is then imaged using a fluorescent microscope and a capture plot is generated. The capture plot shows fluorescent particles and indicates the emission wavelength of the fluorescent particles. The capture plot can be evaluated against a standard result to determine the quality of the reagents and the quality of the microfluidic device for retaining particles or cells displaying EpCAM.

### Example 4

### Sorting Cells Based on Size and Affinity

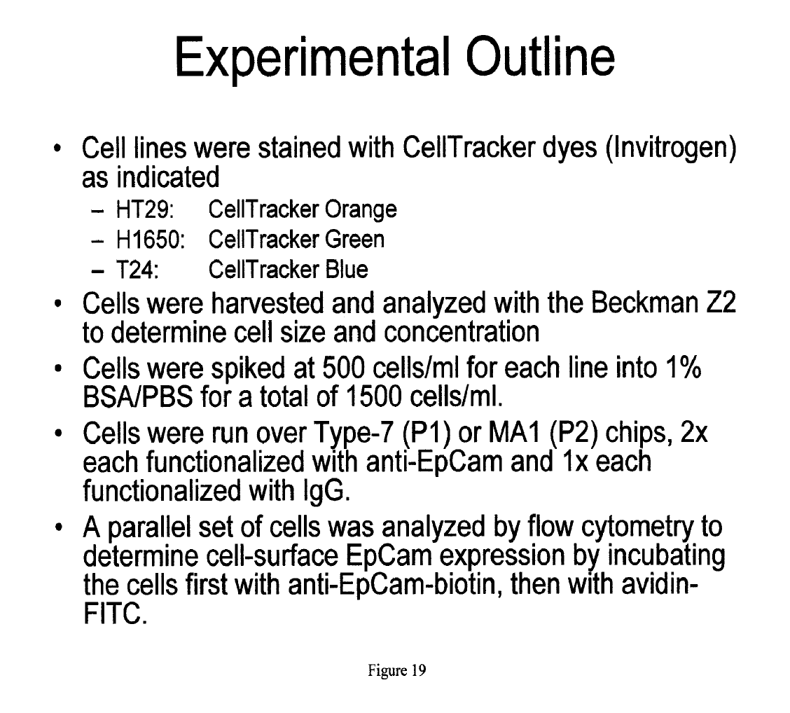

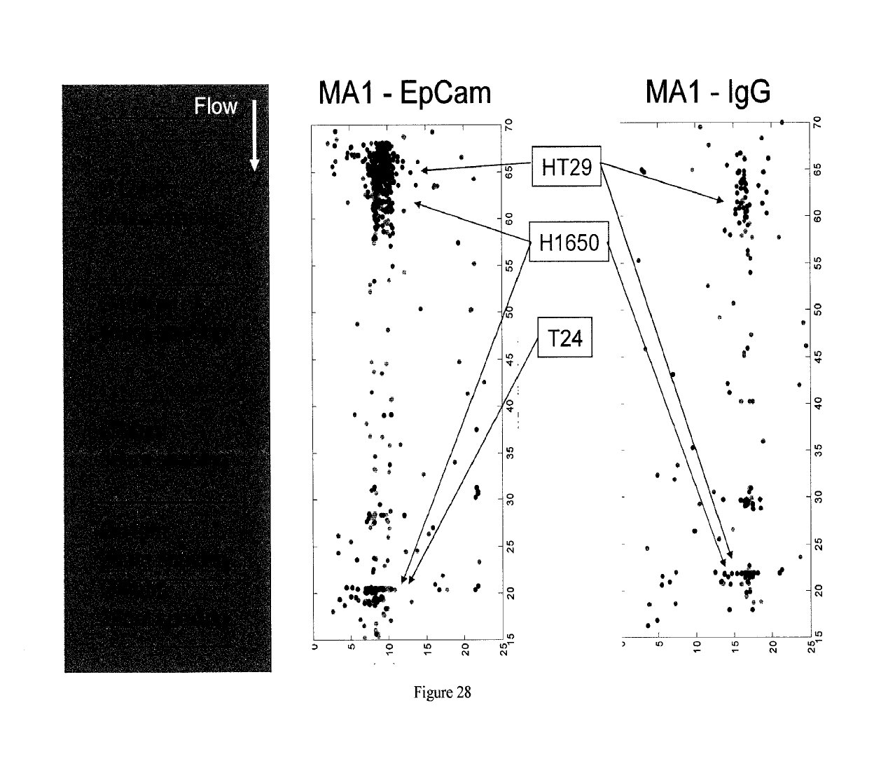

An experimental outline is shown in FIG. 19. Three cell lines with different cell surface markers and size distribution were analyzed using four microfluidic devices. Two microfluidic devices were functionalized with antibodies to EpCAM and two microfluidic devices were functionalized with antibodies to IgG. One of the two microfluidic devices functionalized to either EpCAM or IgG was a T7 chip and the other was a MA1 chip. The T7 chip comprises restricted gaps or pinch points and the MA1 chip comprises five subarrays of decreasing gap length. The gap length or spacing and the direction of sample flow is shown in FIG. 28 for the five subarrays. Obstacle diameter is also indicated by the 0 symbol.

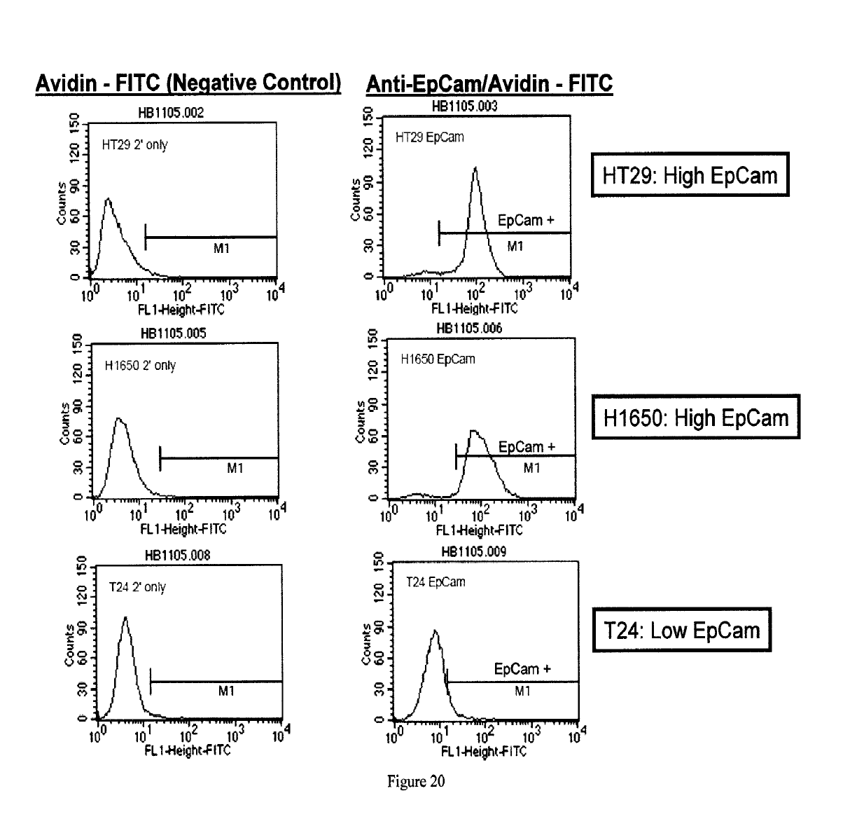

The three cell lines were HT29, which has high EpCAM levels, H1650, which has high EpCAM levels, and T24, which has low EpCAM levels. Levels of EpCAM were evaluated using fluorescently labeled antibodies to EpCAM and, as a negative control, fluorescently labeled antibodies to avidin, as shown in FIG. 20.

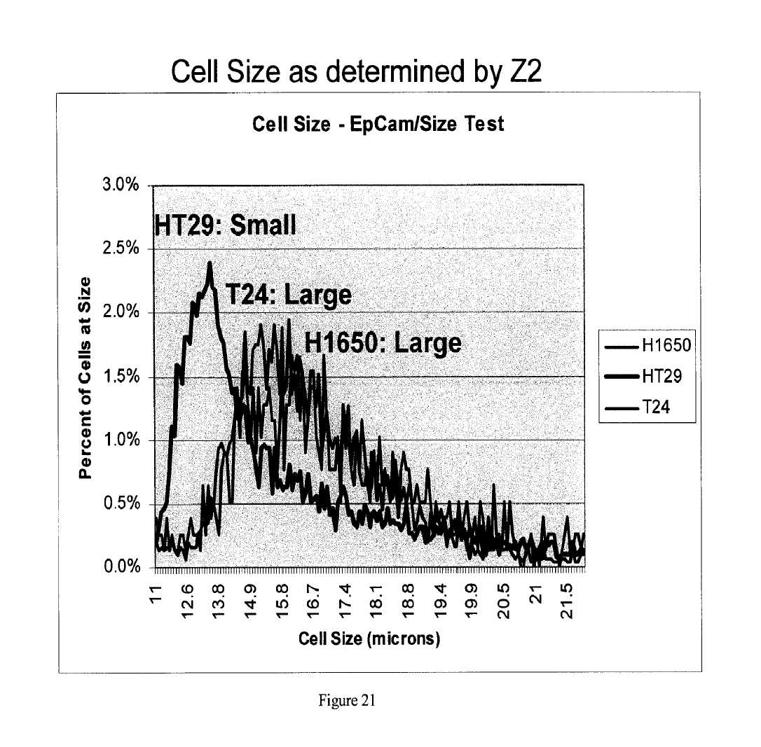

The three cell lines were analyzed using a Beckman Z2 to determine cell size and concentration. As shown in FIG. 21, the H1650 cells and the T24 cells were large and the HT29 cells were small.

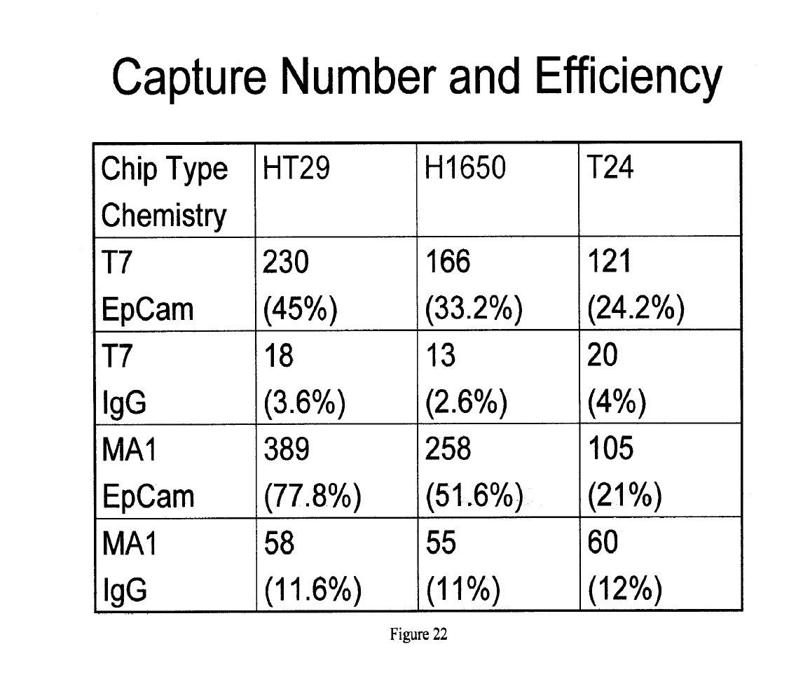

Capture efficiency of the different cell lines through the four microfluidic devices were analyzed. The results are shown in FIG. 22. The number of cells captured is reported in the grid corresponding to a microfluidic chip and a cell line. The value in the parentheses is an indication of capture efficiency.

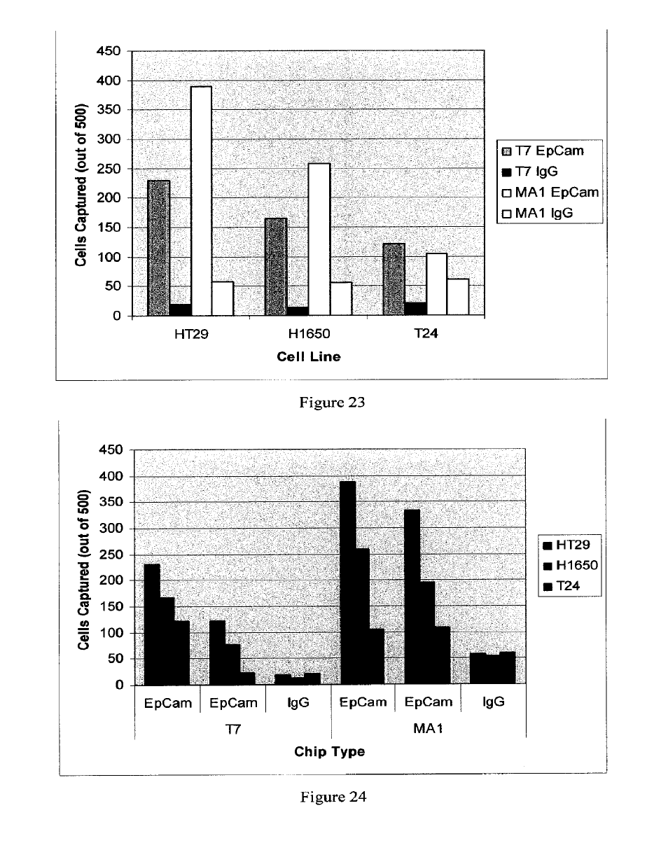

FIG. 23 shows the cells captured as a function of cell type in a graphical layout. FIG. 24 shows cells captured as a function of chip type in a graphical layout. FIG. 25 shows the cells captured as a function of chip type in a graphical layout and showing standard deviation.

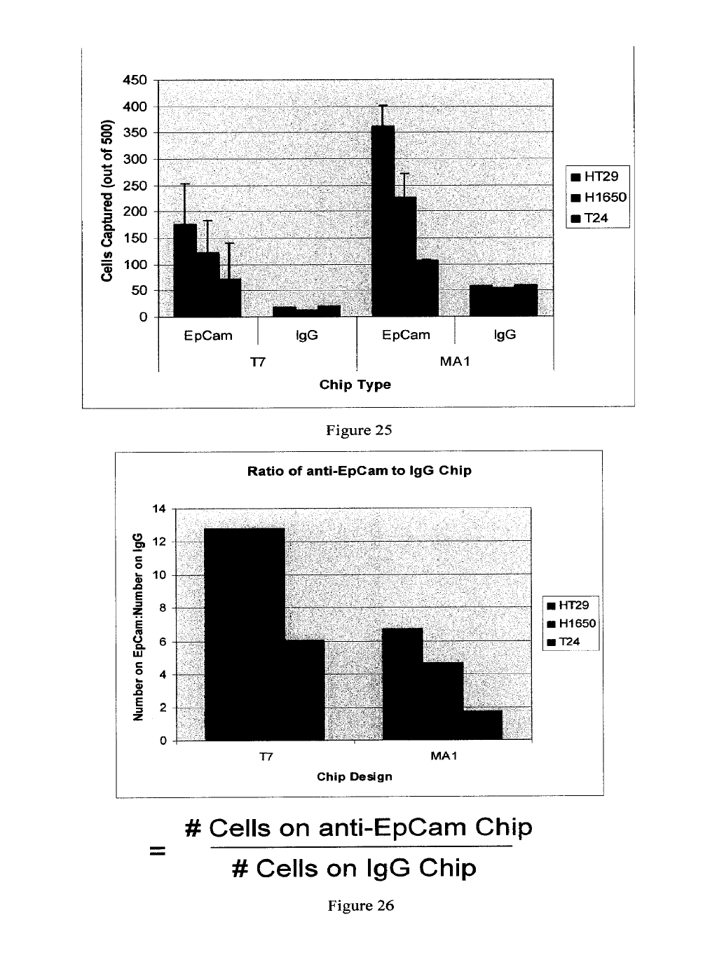

FIG. 26 shows a ratio of the cells captured for an anti-EpCAM chip vs an anti-IgG chip. The T7 anti-IgG chip has a reduced amount of cells captured, thus increasing the amount of cells captured on the anti-EpCAM chip relative to the anti-IgG chip.

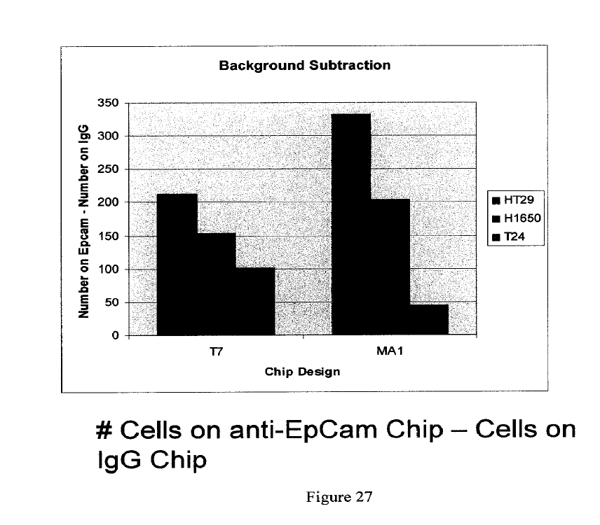

Alternatively, FIG. 27 shows that the relative number of cells captured by anti-EpCAM above the number of cells captured by anti-IgG chips is greater for the MA1 chip.

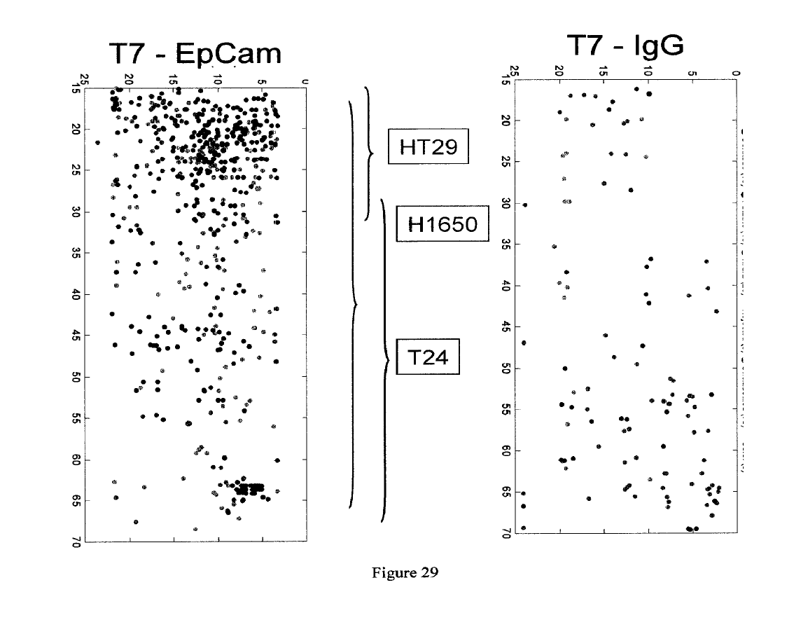

FIG. 28 and FIG. 29 show capture plots indicating spatial localization of cells captured by the MA1 and T7 chips, respectively. The MA1 chips show spatial localization of HT29 cells near the entrance of flow for the MA1-anti-EpCAM chips and the near the entrance and exit of the MA1-anti-IgG chips. While the HT29 cells are small, the anti-EpCAM is able to facilitate binding of HT29 cells.

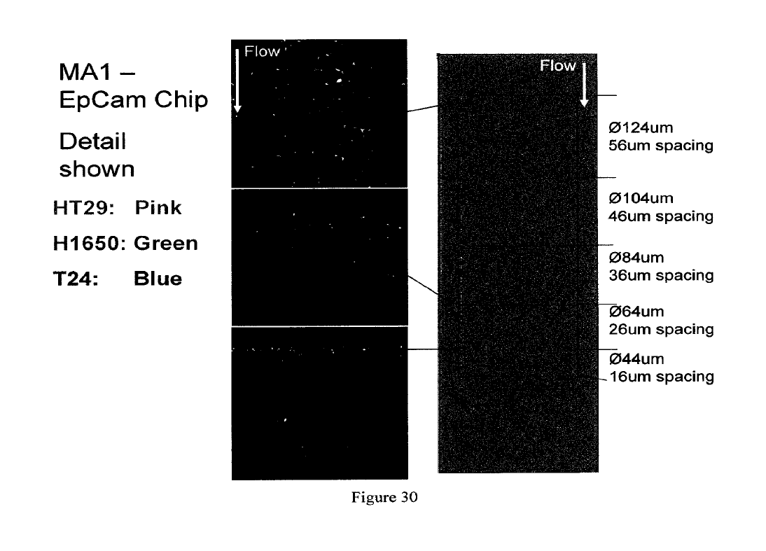

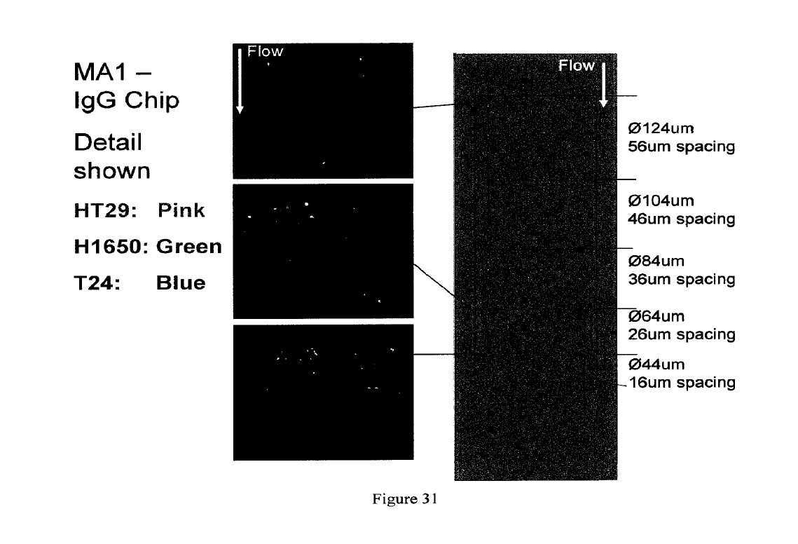

FIG. 30 and FIG. 31 show fluorescence microscope images of cells captured by the MA1-anti-EpCAM and MA1-anti-IgG chips, respectively.

1. A microfluidic device comprising:
an array of obstacles including a first subarray of obstacles and a second subarray of obstacles that are fluidly connected and positioned such that a fluid medium introduced to an inlet of the microfluidic device passes sequentially through the first subarray then the second subarray before exiting through an outlet of the microfluidic device;
wherein the first subarray or the second subarray of obstacles is functionalized with one or more sets of one or more binding moieties.

2. The microfluidic device of claim 1, wherein the sets of one or more binding moieties includes two or more binding moieties.

3. The microfluidic device of claim 1, wherein the first subarray and the second subarray of obstacles are functionalized with one or more sets of one or more binding moieties.

4. The microfluidic device of claim 1, further comprising a first set of one or more binding moieties functionalized in a first region of the first subarray and a second set of one or more binding moieties functionalized in a second region of the first subarray.

5. The microfluidic device of claim 1, further comprising a first set of one or more binding moieties functionalized in a first region of the second subarray and a second set of one or more binding moieties functionalized in a second region of the second subarray.

6. The microfluidic device of claim 4, wherein the first set of one or more binding moieties and the second set of one or more binding moieties include two or more binding moieties.

7. The microfluidic device of claim 4, wherein the first region is distinct from the second region.

8. The microfluidic device of claim 1, wherein the obstacles are fixed to the microfluidic device.

9. The microfluidic device of claim 1, wherein the first subarray has a first average gap length between adjacent obstacles and the second subarray has a second average gap length between adjacent obstacles, wherein the first average gap length is greater than the second average gap length.

10. The microfluidic device of claim 7, wherein the second average gap length is less than 8, 10, 12, 15, 17, 20, 24, 29, 35, or 42 microns.

11. The microfluidic device of claim 1, wherein a sample obtained from a patient is contacted with the microfluidic device and one or more rare cells are retained by the microfluidic device.

12. The microfluidic device of claim 11, wherein 1, 5, or 20% of the one or more rare cells retained by the microfluidic device are retained in the first 30 rows of the second subarray of obstacles.

13. A method for diagnosing cancer comprising enumerating one or more enriched circulating tumor cells and fragments thereof using a bright field microscope.

14. The method of claim 13, wherein the enumerating comprises staining the one or more enriched circulating tumor cells.

15. The method of claim 13, wherein the staining includes an indicator for a cancer marker.

16. The method of claim 15, wherein the cancer marker is cytokeratin, EGFR, EpCAM, cadherin, mucin, or LAR

17. The method of claim 15, wherein the cancer marker is cytokeratin.

18. The method of claim 14, wherein the staining includes using a pan-cytokeratin antibody, a biotinylated secondary antibody, an avidin-biotinylated horseradish peroxidase complex, and diaminobenzidine tetrahydrochloride.

19. The method of claim 18, wherein the pan-cytokeratin antibody is a mixture of monoclonal antibodies.

20. The method of claim 14, wherein the stain includes AE1/AE3 antibodies.

21-100. (canceled)
